SEQUENCE LISTING (73 sequences)

SEQ ID NO: 1 - DNA, 15 bp, synthetic construct
  source 1..15: mol_type = other DNA
ggctagctacaacga

SEQ ID NO: 2 - DNA, 13 bp, synthetic construct
  source 1..13: mol_type = other DNA
ccgagccggacga

SEQ ID NO: 3 - DNA, 14 bp, synthetic construct
  source 1..14: mol_type = other DNA
gtcagcgacacgaa

SEQ ID NO: 4 - DNA, 14 bp, synthetic construct
  source 1..14: mol_type = other DNA
gtcagtgactcgaa

SEQ ID NO: 5 - DNA, 15 bp, synthetic construct
  source 1..15: mol_type = other DNA
gtcagctgactcgaa

SEQ ID NO: 6 - DNA, 20 bp, synthetic construct
  source 1..20: mol_type = other DNA
aggaggtaggggttccgctc

SEQ ID NO: 7-8 - skipped (2 entries)

SEQ ID NO: 9 - DNA, 33 bp, synthetic construct
  source 1..33: mol_type = other DNA
gggaaaggaggctagctacaacgaaagggggag

SEQ ID NO: 10 - DNA, 31 bp, synthetic construct
  source 1..31: mol_type = other DNA
gggaaaggaccgagccggacgaaagggggag

SEQ ID NO: 11 - DNA, 32 bp, synthetic construct
  source 1..32: mol_type = other DNA
gggaaaggagtcagcgacacgaaaagggggag

SEQ ID NO: 12 - DNA, 32 bp, synthetic construct
  source 1..32: mol_type = other DNA
gggaaaggagtcagtgactcgaaaagggggag

SEQ ID NO: 13 - DNA, 33 bp, synthetic construct
  source 1..33: mol_type = other DNA
gggaaaggagtcagctgactcgaaaagggggag

SEQ ID NO: 14 - DNA, 38 bp, synthetic construct
  source 1..38: mol_type = other DNA
gggaaaggaaggaggtaggggttccgctcaagggggag

SEQ ID NO: 15 - DNA, 25 bp, synthetic construct
  source 1..25: mol_type = other DNA
ggaggctagctacaacgaaaggggg

SEQ ID NO: 16 - DNA, 33 bp, synthetic construct
  source 1..33: mol_type = other DNA
atcttccttggctagctacaacgacgcctctcc

SEQ ID NO: 17 - DNA, 28 bp, synthetic construct
  source 1..28: mol_type = other DNA
gggaaaggaggctagctacaacgaaagg

SEQ ID NO: 18 - DNA, 29 bp, synthetic construct
  source 1..29: mol_type = other DNA
gggaaaggaggctagctacaacgaaaggg

SEQ ID NO: 19 - DNA, 30 bp, synthetic construct
  source 1..30: mol_type = other DNA
gggaaaggaggctagctacaacgaaagggg

SEQ ID NO: 20 - DNA, 30 bp, synthetic construct; identical to SEQ ID NO: 19
  source 1..30: mol_type = other DNA

SEQ ID NO: 21 - DNA, 30 bp, synthetic construct; identical to SEQ ID NO: 19
  source 1..30: mol_type = other DNA

SEQ ID NO: 22 - DNA, 27 bp, synthetic construct
  source 1..27: mol_type = other DNA
ggaaaggaggctagctacaacgaaagg

SEQ ID NO: 23 - DNA, 28 bp, synthetic construct
  source 1..28: mol_type = other DNA
ggaaaggaggctagctacaacgaaaggg

SEQ ID NO: 24 - DNA, 29 bp, synthetic construct
  source 1..29: mol_type = other DNA
ggaaaggaggctagctacaacgaaagggg

SEQ ID NO: 25 - DNA, 29 bp, synthetic construct
  source 1..29: mol_type = other DNA
ggaaaggaggctagctacaacgaaagggg

SEQ ID NO: 26 - DNA, 29 bp, synthetic construct
  source 1..29: mol_type = other DNA
ggaaaggaggctagctacaacgaaagggg

SEQ ID NO: 27 - DNA, 30 bp, synthetic construct
  source 1..30: mol_type = other DNA
ggaaaggaggctagctacaacgaaaggggg

SEQ ID NO: 28 - DNA, 30 bp, synthetic construct; identical to SEQ ID NO: 27
  source 1..30: mol_type = other DNA

SEQ ID NO: 29 - DNA, 30 bp, synthetic construct; identical to SEQ ID NO: 27
  source 1..30: mol_type = other DNA

SEQ ID NO: 30 - DNA, 30 bp, synthetic construct; identical to SEQ ID NO: 27
  source 1..30: mol_type = other DNA

SEQ ID NO: 31 - DNA, 30 bp, synthetic construct; identical to SEQ ID NO: 27
  source 1..30: mol_type = other DNA

SEQ ID NO: 32 - DNA, 24 bp, synthetic construct
  source 1..24: mol_type = other DNA
ggaggctagctacaacgaaagggg

SEQ ID NO: 33 - DNA, 25 bp, synthetic construct
  source 1..25: mol_type = other DNA
ggaggctagctacaacgaaaggggg

SEQ ID NO: 34 - DNA, 33 bp, synthetic construct
  source 1..33: mol_type = other DNA
  modified_base 2: mod_base = OTHER; note = 8-aza-7-deazaguanosine
  modified_base 28: mod_base = OTHER; note = 8-aza-7-deazaguanosine
  modified_base 30: mod_base = OTHER; note = 8-aza-7-deazaguanosine
gngaaaggaggctagctacaacgaaagngngag

SEQ ID NO: 35 - skipped

SEQ ID NO: 36 - DNA, 35 bp, synthetic construct
  source 1..35: mol_type = other DNA
  misc_difference 1: note = a, c, g, or t
  misc_difference 2: note = a, c, g, or t
  misc_difference 3: note = a, c, g, or t
  misc_difference 4: note = a, c, g, or t
  misc_difference 5: note = a, c, g, or t
  misc_difference 6: note = a, c, g, or t
  misc_difference 7: note = a, c, g, or t
  misc_difference 8: note = a, c, g, or t
  misc_difference 9: note = a, c, g, or t
  misc_difference 26: note = a, c, g, or t
  misc_difference 27: note = a, c, g, or t
  misc_difference 28: note = a, c, g, or t
  misc_difference 29: note = a, c, g, or t
  misc_difference 30: note = a, c, g, or t
  misc_difference 31: note = a, c, g, or t
  misc_difference 32: note = a, c, g, or t
  misc_difference 33: note = a, c, g, or t
  misc_difference 34: note = a, c, g, or t
  misc_difference 35: note = a, c, g, or t
nnnnnnnnnrggctagctacaacgannnnnnnnnn

SEQ ID NO: 37 - DNA, 33 bp, synthetic construct
  source 1..33: mol_type = other DNA
aggacgggaggctagctacaacgagtggttgcc

SEQ ID NO: 38 - DNA, 33 bp, synthetic construct
  source 1..33: mol_type = other DNA
gtggatggaggctagctacaacgagtcttggag

SEQ ID NO: 39 - DNA, 33 bp, synthetic construct
  source 1..33: mol_type = other DNA
aggagaacaggctagctacaacgagggatgagg

SEQ ID NO: 40 - DNA, 37 bp, synthetic construct
  source 1..37: mol_type = other DNA
aggagaacaggctagctacaacgagggatgaggcggg

SEQ ID NO: 41 - DNA, 33 bp, synthetic construct
  source 1..33: mol_type = other DNA
tgcaatgaaggctagctacaacgatggggaggg

SEQ ID NO: 42 - DNA, 33 bp, synthetic construct
  source 1..33: mol_type = other DNA
ttcacaagaggctagctacaacgaagagggggg

SEQ ID NO: 43 - DNA, 33 bp, synthetic construct
  source 1..33: mol_type = other DNA
ccgtatacaggctagctacaacgatgctgggga

SEQ ID NO: 44 - DNA, 33 bp, synthetic construct; identical to SEQ ID NO: 9
  source 1..33: mol_type = other DNA

SEQ ID NO: 45 - DNA, 33 bp, synthetic construct
  source 1..33: mol_type = other DNA
aggggaggaggctagctacaacgattgacgagt

SEQ ID NO: 46 - DNA, 33 bp, synthetic construct
  source 1..33: mol_type = other DNA
aggggggtaggctagctacaacgacaagagcca

SEQ ID NO: 47 - DNA, 33 bp, synthetic construct
  source 1..33: mol_type = other DNA
ggggagggaggctagctacaacgaggggtggat

SEQ ID NO: 48 - DNA, 33 bp, synthetic construct
  source 1..33: mol_type = other DNA
gaggggccaggctagctacaacgagagggcagg

SEQ ID NO: 49 - DNA, 33 bp, synthetic construct
  source 1..33: mol_type = other DNA
aaagtgcaaggctagctacaacgagaactgggg

SEQ ID NO: 50 - DNA, 33 bp, synthetic construct
  source 1..33: mol_type = other DNA
agggaagggggctagctacaacgacaggtccgc

SEQ ID NO: 51 - DNA, 33 bp, synthetic construct
  source 1..33: mol_type = other DNA
gaggaagggggctagctacaacgacgacaatgc

SEQ ID NO: 52 - DNA, 33 bp, synthetic construct
  source 1..33: mol_type = other DNA
gctgcagggggctagctacaacgatggtgcacg

SEQ ID NO: 53 - DNA, 33 bp, synthetic construct
  source 1..33: mol_type = other DNA
tcacacgggggctagctacaacgacacaaggca

SEQ ID NO: 54 - DNA, 33 bp, synthetic construct
  source 1..33: mol_type = other DNA
ccaggaaggggctagctacaacgaagccgagcc

SEQ ID NO: 55 - DNA, 33 bp, synthetic construct
  source 1..33: mol_type = other DNA
agggccaggggctagctacaacgaccgcgggga

SEQ ID NO: 56 - DNA, 33 bp, synthetic construct
  source 1..33: mol_type = other DNA
cagggctggggctagctacaacgagcacgggac

SEQ ID NO: 57 - DNA, 33 bp, synthetic construct
  source 1..33: mol_type = other DNA
cccgaagggggctagctacaacgactcgtgaca

SEQ ID NO: 58 - DNA, 33 bp, synthetic construct
  source 1..33: mol_type = other DNA
agtccgaaaggctagctacaacgaggcgggggc

SEQ ID NO: 59 - DNA, 33 bp, synthetic construct
  source 1..33: mol_type = other DNA
ggaccgggaggctagctacaacgatatgtctct

SEQ ID NO: 60 - DNA, 33 bp, synthetic construct
  source 1..33: mol_type = other DNA
tgtcgctaaggctagctacaacgaggattgggc

SEQ ID NO: 61 - DNA, 33 bp, synthetic construct
  source 1..33: mol_type = other DNA
ttagctgtaggctagctacaacgacgtcaaggc

SEQ ID NO: 62 - DNA, 33 bp, synthetic construct
  source 1..33: mol_type = other DNA
ggggagagaggctagctacaacgagggccctca

SEQ ID NO: 63 - DNA, 33 bp, synthetic construct
  source 1..33: mol_type = other DNA
ggtcgtataggctagctacaacgacaaaggcct

SEQ ID NO: 64 - DNA, 33 bp, synthetic construct
  source 1..33: mol_type = other DNA
tgtcgagaaggctagctacaacgaatccaagag

SEQ ID NO: 65 - DNA, 33 bp, synthetic construct
  source 1..33: mol_type = other DNA
ggtccctcaggctagctacaacgatgcactgta

SEQ ID NO: 66 - DNA, 33 bp, synthetic construct
  source 1..33: mol_type = other DNA
agggggcaaggctagctacaacgagtcggggca

SEQ ID NO: 67 - DNA, 33 bp, synthetic construct
  source 1..33: mol_type = other DNA
aagcggggaggctagctacaacgactgggcgga

SEQ ID NO: 68 - DNA, 33 bp, synthetic construct
  source 1..33: mol_type = other DNA
gggtcgtcaggctagctacaacgactggctccc

SEQ ID NO: 69 - DNA, 33 bp, synthetic construct
  source 1..33: mol_type = other DNA
atgcatctaggctagctacaacgacaaaaaaaa

SEQ ID NO: 70 - DNA, 33 bp, synthetic construct
  source 1..33: mol_type = other DNA
cctcggccaggctagctacaacgaccgctccgg

SEQ ID NO: 71 - DNA, 33 bp, synthetic construct
  source 1..33: mol_type = other DNA
gctgctcgaggctagctacaacgaggcacggcg

SEQ ID NO: 72 - DNA, 33 bp, synthetic construct
  source 1..33: mol_type = other DNA
caggtgtgaggctagctacaacgaagataagga

SEQ ID NO: 73 - DNA, 33 bp, synthetic construct
  source 1..33: mol_type = other DNA
gttcctctaggctagctacaacgaggggtcgtc

(19) United States
(12) Patent Application Publication
(10) Pub. No.: US 2024/0344064 A1
(43) Pub. Date: Oct. 17, 2024

(54) GUANINE-RICH DEOXYRIBOZYMES, COMPOSITIONS AND USES THEREOF

(71) Applicant: 1E Therapeutics, Ltd., Rehovot (IL)

(72) Inventors: Ido BACHELET, Rehovot (IL); Almogit ABU-HOROWITZ, Rehovot (IL); Gat KRIEGER, Rehovot (IL); Yossi OVADYA, Rehovot (IL); Dina RAICHLIN, Rehovot (IL); Etti KATZ-KADOSH, Rehovot (IL); Alaa KNANY, Rehovot (IL); Shireen BARAKEY, Rehovot (IL); Noam BOROVSKY, Rehovot (IL); Noam COHEN, Rehovot (IL); Hagai MARMOR-KOLLET, Rehovot (IL); Alexander ROSENBERG, Rehovot (IL); Adva LEVY-ZAMIR, Rehovot (IL)

(73) Assignee: 1E Therapeutics, Ltd., Rehovot (IL)

(21) Appl. No.: 18/294,220

(22) PCT Filed: Aug. 4, 2022
(86) PCT No.: PCT/IL2022/050848

§ 371 Date: Feb. 1, 2024

Related U.S. Application Data
(60) Provisional application No. 63/229,327, filed on Aug. 4, 2021.

(51) Int. Cl. C12N 15/113; A61P 35/00

(52) U.S. Cl. CPC C12N 15/113; A61P 35/00; C12N 2310/127

(57) ABSTRACT

Provided herein are modified DNAzymes, including DNAzymes with overhang sequences not complementary to the target RNA, in particular, overhangs with high G content. Also provided are compositions comprising the DNAzyme; vectors including the DNAzymes; pharmaceutical compositions including the vectors; and methods including any of the above for cleaving a target RNA; inducing cell death; and treating various diseases and disorders.

### BACKGROUND

Ribozymes and deoxyribozymes (or DNAzymes), are catalytically-active DNA molecules (Breaker, R. R. & Joyce, G. F. (1994) Chemistry & Biology 1:223-229; Silverman, S. K. (2005) Nucleic Acids Res. 33:6151-6163). While catalytic RNA molecules (ribozymes) exist naturally and are involved in basic biological processes such as splicing (Villa, T., Pleiss, J. A. & Guthrie, C. (2002) Cell 109:149-152), translation (Ban, N., Nissen, P., Hansen, J., Moore, P. B. & Steitz, T. A. (2000) Science 289:905-920), and viral self-processing (Doudna, J. A. & Cech, T. R. (2002) Nature 418:222-228), DNAzymes have not been reported in nature.

DNAzymes are typically generated by in-vitro selection, and several types have already been isolated and characterized (Breaker, R. R. & Joyce, G. F. (1994) Chemistry & Biology 1:223-229; Breaker, R. R. & Joyce, G. F. (1995) Chemistry & Biology 2:655-660; Santoro, S. W. & Joyce, G. F. (1997) Proceedings of the National Academy of Sciences 94:4262-4266; Geyer, C. R. & Sen, D. (1997) Chem. Biol. 4:579-593; Roth, A. & Breaker, R. R. (1998) Proceedings of the National Academy of Sciences 95:6027-6031). Ribozymes and DNAzymes are diverse structurally and mechanistically, and exhibit diverse secondary structures, metal ion dependencies, and catalysis kinetics (McManus, S. A. & Li, Y. (2010) Molecules 15:6269-6284).

### SUMMARY

In certain aspects, provided herein are DNAzymes comprising 5′ and/or 3′ overhang sequences, wherein said overhangs comprise at least 50% G content, and said DNAzymes target a RNA transcript. In related aspects, disclosed herein are nucleic acids and vectors encoding such DNAzymes, pharmaceutical compositions comprising such DNAzymes, as well as libraries enriched with such DNAzymes. In some aspects, provided herein are methods of screening for active DNAzymes, methods of using DNAzymes, nucleic acids, vectors and/or pharmaceutical compositions described herein for cleaving a RNA transcript or inhibiting expression of a gene. In certain embodiments, the RNA transcript is an mRNA encoding p21, USP7, KRAS, or BIRC5.

In certain aspects, provided herein are DNAzymes targeting a RNA transcript, the DNAzyme comprising, in 5′ to 3′ order: (i) a first substrate-binding domain (also referred to herein as the “5′ arm”) comprising a sequence that base pairs with a first region of the RNA transcript; (ii) a DNAzyme catalytic core; and (iii) a second substrate-binding domain (also referred to herein as the “3′ arm”) comprising a sequence that base pairs with a second region of the RNA transcript positioned 5′ to the first region of the RNA transcript, said DNAzyme further comprises at least one of: (a) a 5′ overhang sequence having at least 50% G content that is 5′ to the first substrate binding domain or a 3′ overhang sequence having at least 50% G content that is 3′ to the second substrate binding domain, wherein upon binding of the DNAzyme to the RNA transcript, the DNAzyme catalytic core cleaves the RNA transcript at a position between the first and second region of the RNA transcript, In further related aspects, at least 30% (e.g., at least 35%, at least 40%, at least 45%, at least 50%, at least 55%, at least 60%, at least 65%, at least 70%, at least 75%, at least 80%, at least 85%, at least 90%, at least 95%, or at least 99%) of the nucleotides in the first substrate-binding domain and/or the second substrate-binding domain are guanine (G). In another further related aspect, at least 60% of the nucleotides in the combined sequences of the 5′ and 3′ arms are G. In yet another further related aspect, at least 70%, at least 80%, at least 90%, or 100% of the nucleotides in the combined sequences of the 5′ and 3′ arms are G.

In a related aspect, at least 30% (e.g., at least 35%, at least 40%, at least 45%, at least 50%, at least 55%, at least 60%, at least 65%, at least 70%, at least 75%, at least 80%, at least 85%, at least 90%, at least 95%, or 100%) of the nucleotides in the 5′ extension are G, and the 5′ extension does not base pair with the RNA transcript. In another related aspect, at least 30% (e.g., at least 35%, at least 40%, at least 45%, at least 50%, at least 55%, at least 60%, at least 65%, at least 70%, at least 75%, at least 80%, at least 85%, at least 90%, at least 95%, or 100%) of the nucleotides in the 3′ extension are G, and the 3′ extension does not base pair with the RNA transcript. Extensions that are not complementary to the RNA target are also referred to herein as “overhang[s]”.

In a related aspect, the DNAzyme catalytic core is a 10-23 catalytic core, a 8-17 catalytic core, a E1111 catalytic core, a E2112 catalytic core, a E5112 catalytic core, or a bipartite catalytic core. In further related aspects, the DNAzyme catalytic core is a 10-23 catalytic core. another further related aspect, the DNAzyme catalytic core comprises a nucleic acid sequence selected from any one of SEQ ID NOs: 1-6. In another further related aspect, the DNAzyme catalytic core comprises the nucleic acid sequence of SEQ ID NO: 1.

In a related aspect, the 5′ arm is any of the lengths or length ranges mentioned herein. In another related aspect, the 3′ arm is any of the lengths or length ranges mentioned herein. In yet another related aspect, the 5′ arm and the 3′ arm are independently selected from any of the lengths or length ranges mentioned herein.

In a related aspect, the 5′ arm is fully complementary to the first region of the RNA transcript or partially complementary to the first region of the RNA transcript with no more than two mismatches. In another related aspect, the 3′ arm is fully complementary to the second region of the RNA transcript or partially complementary to the second region of the RNA transcript with no more than two mismatches. In another related aspect, the 5′ arm and the 3′ arm together have no more than 3 mismatches to the first and second regions of the RNA transcript.

In a related aspect, the RNA transcript is from a prokaryotic gene. In another related aspect, the RNA transcript is from a eukaryotic gene. In another related aspect, the RNA transcript is a messenger RNA (mRNA) or a pre-messenger RNA (pre-mRNA) of a eukaryotic gene. In another related aspect, the RNA transcript is a P21 mRNA. In other related aspects, the RNA transcript is USP7, KRAS, or BIRC5, as defined herein

In a related aspect, the 5′ arm comprises the nucleic acid sequence 5′-GGGAAAGGA-3′ (SEQ ID NO: 7), and the 3′ arm comprises the nucleic acid sequence 5′-AAGGGGGAG-3′ (SEQ ID NO: 8). In other related aspects, the DNAzyme comprises a nucleic acid sequence selected from SEQ ID NOs: 9-14. In another related aspect, the DNAzyme comprises the nucleic acid sequence of SEQ ID NO: 9.

In a related aspect, the DNAzyme further comprises a 5′ overhang immediately 5′ to the 5′ end of the 5′ arm, and a 3′ overhang immediately 3′ to the 3′ end of the 3′ arm, wherein at least 30% (e.g., at least 35%, at least 40%, at least 45%, at least 50%, at least 55%, at least 60%, at least 65%, at least 70%, at least 75%, at least 80%, at least 85%, at least 90%, at least 95%, or 100%) of the total nucleotides in the 2 overhangs are G. In a further related aspect, 5′ and 3′ overhangs are both present, and at least 60% of the nucleotides in the combined sequences of the 5′ and 3′ overhangs are G. In another further related aspect, at least 70%, at least 80%, at least 90%, or 100% of the nucleotides in the combined sequences are G.

In a related aspect, the 5′ extension is 2 to 10 nucleotides in length. In another related aspect, the 3′ extension is 2 to 10 nucleotides in length, and the 5′ extension is 1 to 10 nucleotides in length.

In a related aspect, the 5′ arm and the 3′ arm form a structure within the domain itself, or forms a structure with each other. In another related aspect, the 5′ extension and the 3′ extension form a structure within the extension itself, or forms a structure with each other. In another related aspect, the structure does not interfere with the binding or the cleavage of the RNA transcript. In yet another related aspect, the structure is a structure formed between DNA strands based on Hoogsteen or wobble base pairing. In a further related aspect, the structure is a G-quadruplex, G-triplex, or H-DNA. In yet a further related aspect, the structure is an intramolecular structure or an intermolecular structure formed by up to 4 nucleotides.

In a related aspect, the 5′ extension and the 3′ extension are independently selected from any of the lengths or ranges of lengths mentioned herein.

In a related aspect, the DNAzyme catalytic core is a 10-23 catalytic core, a 8-17 catalytic core, a E1111 catalytic core, a E2112 catalytic core, a E5112 catalytic core, or a bipartite catalytic core. In a further related aspect, the DNAzyme catalytic core is a 10-23 catalytic core. In further related aspects, the DNAzyme catalytic core comprises a nucleic acid sequence selected from any one of SEQ ID NOs: 1-6. In yet a further related aspect, the DNAzyme catalytic core comprises a nucleic acid sequence of SEQ ID NO: 1.

In a related aspect, the 5′ arm is 6-15 nucleotides in length. In a further related aspect, the 5′ arm is 6-12 nucleotides in length. In another further related aspect, the 5′ arm is 6-11, 6-10, 7-12, 7-11, 7-10, 8-12, 8-11, 8-10, 8-9, or 9-10 nucleotide in length. In another further related aspect, the 5′ arm is 9 nucleotides in length. In a related aspect, the 3′ arm is 6-15 nucleotides in length. In another further related aspect, the 3′ arm is 6-12 nucleotides in length. In another further related aspect, the 3′ arm is 6-11, 6-10, 7-12, 7-11, 7-10, 8-12, 8-11, 8-10, 8-9, or 9-10 nucleotide in length. In yet another further related aspect, the 3′ arm is 9 nucleotides in length. In a related aspect, the 5′ arm and the 3′ arm are independently selected from any of the aforementioned ranges. In a further related aspect, the 5′ arm and the 3′ arm are each 8, 9, or 10 nucleotides in length.

In a related aspect, the target RNA transcript is from a prokaryotic gene. In a related aspect, the RNA transcript is from a eukaryotic gene. In a further related aspect, the RNA transcript is a messenger RNA (mRNA) or a pre-messenger RNA (pre-mRNA) of a eukaryotic gene.

In a related aspect, the 5′ and/or 3′ overhang comprise(s) at least one tandem repeat of G, having 2 or more consecutive Gs. In a further related aspect, the 5′ and/or 3′ overhang comprise(s) at least one tandem repeat of at least 3 G's. In another further related aspect, the 5′ and/or 3′ overhang comprise(s) at least one tandem repeat of at least 4 G's. In another further related aspect, the 5′ and/or 3′ overhang comprise(s) at least one tandem repeat of at least 5 G's. In yet another further related aspect, the 5′ and/or 3′ overhang comprises at least one guanine (G) at every interval of 3 nucleotides.

In a related aspect, the DNAzyme comprises a chemical modification, which may be any modification mentioned herein.

In a related aspect, the DNAzyme comprises ribonucleotides, deoxyribonucleotides, or a combination thereof.

In a related aspect, a described DNAzyme exhibits one or more effects on a target cells independent of cleavage of the target RNA. In a further related aspect, the effects comprise induction of cell death. In another further related aspect, off-target effects may synergize with effects mediated by cleavage of the target RNA.

In certain aspects, provided herein are nucleic acids comprising one or more DNAzymes described herein. In a related aspect, each DNAzyme sequence is operably linked to an origin of replication, and is replicated inside bacteria to produce additional DNAzymes. Non-limiting examples of replicating DNAzymes are replicating circular DNAzymes, e.g., as described in Chen F, et al., A novel replicating circular DNAzyme. Nucleic Acids Res. 2004 Apr. 28; 32(8):2336-41.

In a related aspect, the nucleic acid is operably linked to an origin of replication and to a termination site, and wherein the nucleic acid comprises a cleavable sequence between each DNAzyme. In a further related aspect, the nucleic acid is replicated to produce one or more DNAzymes.

In certain aspects, provided herein are vectors comprising the nucleic acids described herein. In further related aspects, the vectors are configured to produce the DNAzymes described herein.

In certain aspects, provided herein are pharmaceutical compositions comprising the DNAzymes described herein. In certain aspects, provided herein are pharmaceutical compositions comprising the vectors described herein. In related aspects, the pharmaceutical compositions described herein further comprise a pharmaceutically acceptable carrier.

In certain aspects, provided herein are libraries of DNAzymes, wherein at least 30% (e.g., at least 35%, at least 40%, at least 45%, at least 50%, at least 55%, at least 60%, at least 65%, at least 70%, at least 75%, at least 80%, at least 85%, at least 90%, at least 95%, or at least 99%, or 100%) of the DNAzymes in the library are the described DNAzymes. In related aspects, all of the DNAzymes in the library are guanine-rich DNAzymes described herein. In a further related aspect, the library of DNAzymes described herein comprises 10²-10¹⁵ unique DNAzymes.

In certain aspects, provided herein are methods of screening for a DNAzyme that cleaves a RNA transcript, comprising: (1) providing a library of DNAzymes described herein; (2) incubating the library of DNAzymes with the RNA transcript; (3) detecting the cleavage of the RNA transcript by one or more DNAzymes from the library. In a related aspect, the method is a cell-free assay. In a further related aspect, the method is a cell-based assay. In yet another further related aspect, the detection method is a cell death assay.

In certain aspects, provided herein are methods of cleaving a RNA transcript, comprising contacting the RNA transcript with a DNAzyme described herein.

In certain aspects, provided herein are methods of inhibiting expression of a gene, comprising contacting a RNA transcript of the gene with a DNAzyme described herein.

In a related aspect, the target RNA transcript is from a prokaryotic gene. In another related aspect, the RNA transcript is from a eukaryotic gene. In a further related aspect, the RNA transcript is a messenger RNA (mRNA) or a pre-messenger RNA (pre-mRNA) of a eukaryotic gene.

### BRIEF DESCRIPTION OF THE DRAWINGS

The subject matter regarded as the guanine-rich deoxyribozymes (DNAzymes) disclosed herein is particularly pointed out and distinctly claimed in the concluding portion of the specification. The guanine-rich DNAzymes, however, both as to organization and method of operation, together with objects, features, and advantages thereof, may best be understood by reference to the following detailed description when read with the accompanying drawings in which:

FIG. 1 shows a schematic illustration of the binding of a DNAzyme to a target RNA. 10-23 DNAzyme is depicted, but other catalytic cores bind similarly. The first and second substrate binding domains are illustrated as domain 1 and domain 2. The first and second regions of the target RNA comprise those regions to the left and right of the arrow, respectively. The nucleotide sequences of the target RNA is set forth in SEQ ID NO: 35. The nucleotide sequences of the 10-23 DNAzyme is set forth in SEQ ID NO: 36.

FIGS. 2A-2D show G-rich DNAzyme are efficient in inducing cell death and RNA cleavage in vitro. FIGS. 2A-2D show that efficient DNAzymes were G-enriched. Heatmaps present position-specific frequency matrix of the mRNA target sequence (FIGS. 2A and 2B) and of the DNAzyme sequence (FIGS. 2C and 2D) of type 10-23 DNAzymes, targeting P21 mRNA. FIGS. 2A and 2C present nucleotide frequency in all screened DNAzymes. FIGS. 2B and 2D present nucleotide frequency of 10% most efficient DNAzymes for inducing cell death.

FIG. 3 shows that guanine content was correlated with efficiency of inducing cell death. The nucleotide frequency in the DNAzyme sequence versus killing efficiency is shown. Pearson correlation coefficients is indicated.

FIG. 4 shows that temperature of melting (Tₘ) was not correlated with killing efficiency. The Tₘ computed for the base-paring of both arms of the DNAzyme with its target mRNA is shown.

FIG. 5 shows correlation of guanine content with in vitro cleavage efficiency. In vitro cleavage was tested using gel electrophoresis.

FIG. 6 shows that tandem repeats of Guanine are correlated with killing efficiency. The longest stretch of G repeats per DNAzyme was shown. Graphs represent different human target genes and the cell lines tested. Presents data from P21 (RAC1) Activated Kinase 1 (P21) in senescent IMR-90 (primary human lung fibroblast) cells, Ubiquitin Specific Peptidase 7 (USP7) in human fibroblast BJ cells, KRAS Proto-Oncogene, GTPase (KRAS) in human pancreatic epithelioid carcinoma PANC1 cells, and Baculoviral IAP Repeat Containing 5 (BIRC5) targets in human pancreatic epithelioid carcinoma PANC1 cells and human lung epithelial carcinoma A549 cells. The nucleotide sequences of the p21 DNAzymes are set forth in SEQ ID NOs: 41-49. The nucleotide sequences of the USP7, DRAS, and BIRC5 DNAzymes are provided in Table 5.

FIG. 7 are graphs showing percent (prc) cell death induction (vertical axis) vs. number of G tandem repeats (horizontal axis) in 5′ and 3′ arms (upper left and right panels) and percent (prc) cell death induction (vertical axis) vs. G content (horizontal axis) in 5′ and 3′ arms (lower left and right panels).

FIG. 8 shows G-enriched DNAzymes in progressive clinical stages. GATA3 sequence is from Sel et al. 2008 (Sel et al. (2008) Journal of Allergy and Clinical Immunology 121:910-916.e5).

FIGS. 9A-9C show data evaluating the G-quadruplex contribution to P21_199 induced cell death of cancer cell lines. (FIG. 9A) Intracellular visualization of G-quadruplex structures by BG4 antibody (green) of DNAzyme (red), nuclei are stained with DAPI (blue). (FIG. 9B) Killing effect of P21-199 on human A459 cells (FIG. 9C) Killing effect of P21-199 on murine Neuro-2a cells.

FIG. 10 is a graph showing an increased cell death percentage (vertical axis) induces by a DNAzyme with a G-rich 3′ overhang (right bars) vs. the unmodified DNAzyme (left bars). Growing and senescent cell data are shown in light gray and black bars, respectively.

### DETAILED DESCRIPTION

In the following detailed description, numerous specific details are set forth in order to provide a thorough understanding of the guanine-rich DNAzymes. However, it will be understood by those skilled in the art that the guanine-rich DNAzymes and uses thereof may be practiced without these specific details. In other instances, well-known methods, procedures, and components have not been described in detail so as not to obscure the guanine-rich DNAzymes presented herein.

In certain embodiments, provided herein are guanine-rich DNAzymes targeting a RNA transcript, for example but not limited to targeting a P21, USP7, KRAS, or BIRC5 mRNA transcript, respectively; nucleic acids and vectors encoding guanine-rich DNAzymes; pharmaceutical compositions comprising guanine-rich DNAzymes, as well as libraries composed of, or in other embodiments enriched with, such guanine-rich DNAzymes. In some embodiments, provided herein are methods of screening for active DNAzymes, methods of using guanine-rich DNAzymes, nucleic acids, vectors and/or pharmaceutical compositions described herein for cleaving a RNA transcript or inhibiting expression of a gene.

In some embodiments, disclosed herein are guanine-rich DNAzymes that bind to and cleave a RNA transcript, for example but not limited to a P21, USP7, KRAS, or BIRC5 mRNA transcript, respectively, wherein binding and resultant cleavage of the transcript inhibits expression of a gene. In some embodiments, disclosed herein are nucleic acids or vectors encoding such guanine-rich DNAzymes. In some embodiments, disclosed herein are pharmaceutical compositions comprising guanine-rich DNAzymes, nucleic acids or vectors. In some embodiments, disclosed herein are libraries composed of, or in other embodiments enriched with such guanine-rich DNAzymes, and methods of screening for active DNAzymes with such libraries. In some embodiments, provided herein are methods of using such guanine-rich DNAzymes to cleave a RNA transcript and/or inhibit a gene. In some embodiments, the inhibitory effect or killing effect produced by the guanine-rich DNAzyme is not mediated by cleavage of the mRNA. In some embodiments, provided herein are methods of treating a disease or condition, wherein the method comprises administering a guanine-rich DNAzyme as disclosed herein to a subject in need. In some embodiments, the treated disease or condition is cancer.

Those skilled in the art will appreciate, in light of the present disclosure, that the effect of cleaving an RNA (e.g., an RNA transcript) will depend on the function of the RNA or encoded protein within the cell; non-limiting examples of such scenarios are described herein. In some embodiments, e.g., in the case of an RNA encoding an essential protein, the cell may die upon cleaving the transcript. In other embodiments, e.g., in the case of an RNA that confers a phenotype, cleaving the RNA will suppress the phenotype.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, less than 1 in 100 nucleotides, less than 1 in 200 nucleotides, less than 1 in 500 nucleotides, less than 1 in 1000 nucleotides, less than 1 in 5,000 nucleotides, or less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

In some embodiments, a DNAzyme provided herein comprises, in 5′ to 3′ order: (i) a 5′ arm comprising a sequence that base pairs with a first region of an RNA transcript; (ii) a DNAzyme catalytic core; and (iii) a 3′ arm comprising a sequence that base pairs with a second region of the RNA transcript positioned 5′ to the first region of the RNA transcript, wherein upon binding of the DNAzyme to the RNA transcript, the DNAzyme catalytic core cleaves the RNA transcript at a position between the first and second region of the RNA transcript. In certain embodiments, a guanine-rich DNAzyme provided herein comprises, in 5′ to 3′ order: (i) a 5′ arm comprising a sequence that base pairs with a first region of an RNA transcript; (ii) a DNAzyme catalytic core; and (iii) a 3′ arm comprising a sequence that base pairs with a second region of the RNA transcript positioned 5′ to the first region of the RNA transcript, wherein upon binding of the guanine-rich DNAzyme to the RNA transcript, the DNAzyme catalytic core cleaves the RNA transcript at a position between the first and second region of the RNA transcript, and wherein at least 30% of the nucleotides in the 5′ arm and/or the 3′ arm are guanine (G).

In some embodiments, a DNAzyme provided herein comprises, in 5′ to 3′ order: (i) a 5′ arm comprising a sequence that base pairs with a first region of an RNA transcript; (ii) a DNAzyme catalytic core; and (iii) a 3′ arm comprising a sequence that base pairs with a second region of the RNA transcript positioned 5′ to the first region of the RNA transcript. In certain embodiments, a guanine-rich DNAzyme provided herein comprises, in 5′ to 3′ order: (i) a 5′ arm comprising a sequence that base pairs with a first region of an RNA transcript; (ii) a DNAzyme catalytic core; and (iii) a 3′ arm comprising a sequence that base pairs with a second region of the RNA transcript positioned 5′ to the first region of the RNA transcript, wherein at least 30% of the nucleotides in the 5′ arm and/or the 3′ arm are guanine (G).

In some embodiments, a DNAzyme provided herein comprises, in 5′ to 3′ order: (i) a 5′ overhang sequence having at least 50% G content; (ii) a first substrate-binding domain comprising a sequence that base pairs with a first region of the RNA transcript; (iii) a DNAzyme catalytic core; (iv) a second substrate-binding domain comprising a sequence that base pairs with a second region of the RNA transcript positioned 5′ to the first region of the RNA transcript; and (v) a 3′ overhang sequence having at least 50% G content; wherein upon binding of the DNAzyme to the RNA transcript, the DNAzyme catalytic core cleaves the RNA transcript at a position between the first and second region of the RNA transcript. In some embodiments, a DNAzyme provided herein comprises, in 5′ to 3′ order: (i) a 5′ overhang sequence having at least 50% G content; (ii) a first substrate-binding domain comprising a sequence that base pairs with a first region of the RNA transcript; (iii) a DNAzyme catalytic core; (iv) a second substrate-binding domain comprising a sequence that base pairs with a second region of the RNA transcript positioned 5′ to the first region of the RNA transcript; and (v) a 3′ overhang sequence having at least 50% G content.

In some embodiments, a guanine-rich DNAzyme provided herein comprises, in 5′ to 3′ order: (i) a 5′ overhang sequence having at least 50% G content; (ii) a first substrate-binding domain comprising a sequence that base pairs with a first region of the RNA transcript; (iii) a DNAzyme catalytic core; (iv) a second substrate-binding domain comprising a sequence that base pairs with a second region of the RNA transcript positioned 5′ to the first region of the RNA transcript; and (v) a 3′ overhang sequence having at least 50% G content; wherein at least 30% of the nucleotides in the 5′ arm and/or the 3′ arm are guanine (G), and wherein upon binding of the guanine-rich DNAzyme to the RNA transcript, the guanine-rich DNAzyme catalytic core cleaves the RNA transcript at a position between the first and second region of the RNA transcript. In some embodiments, a guanine-rich DNAzyme provided herein comprises, in 5′ to 3′ order: (i) a 5′ overhang sequence having at least 50% G content; (ii) a first substrate-binding domain comprising a sequence that base pairs with a first region of the RNA transcript; (iii) a DNAzyme catalytic core; (iv) a second substrate-binding domain comprising a sequence that base pairs with a second region of the RNA transcript positioned 5′ to the first region of the RNA transcript; and (v) a 3′ overhang sequence having at least 50% G content 3′ to said second substrate binding domain, wherein at least 30% of the nucleotides in the 5′ arm and/or the 3′ arm are guanine (G).

### Guanine-Rich DNAzymes

DNAzymes are nucleic acids that bind to and cleave RNA targets. In general, a DNAzyme has as structure that includes, in 5′ to 3′ order: (i) a 5′ arm comprising a sequence that base pairs with a first region of an RNA transcript; (ii) a DNAzyme catalytic core; and (iii) a 3′ arm comprising a sequence that base pairs with a second region of the RNA transcript positioned 5′ to the first region of the mRNA. Upon binding of the DNAzyme to the RNA transcript, the DNAzyme catalytic core cleaves the RNA at a position between the first and second region of the RNA transcript.

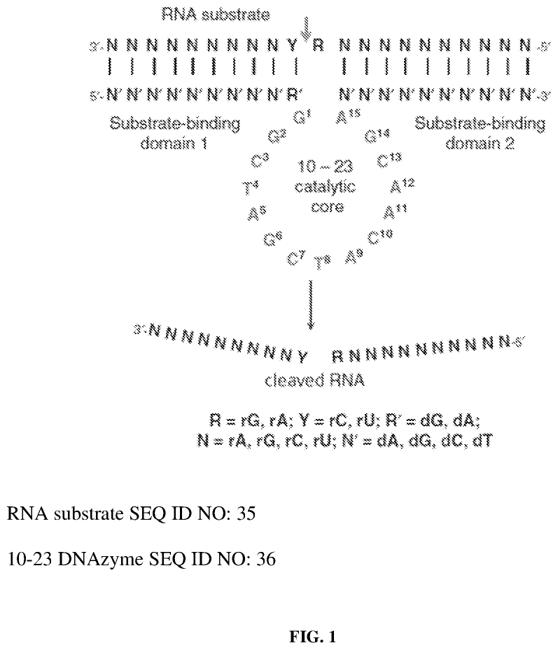

The structure of one embodiment of a DNAzyme is illustrated in FIG. 1, which shows a 10-23 type DNAzyme and its RNA target. The substrate-binding domains of the DNAzyme can include DNA nucleotides, RNA nucleotides, or a combination of DNA and RNA nucleotides. In some embodiments, the described guanine-rich DNAzymes have either or both substrate-binding domains in which at least 30% of nucleotides are guanine (G), and/or have a 5′ and/or a 3′ overhang in which at least 30% of nucleotides are G.

As used herein, the terms “deoxyribozyme” and “DNAzyme” may be used interchangeably having the same qualities and meaning, wherein a DNAzyme encompasses a single stranded oligonucleotides designed to hybridize with a target RNA and cleave it at a specific site.

A skilled artisan would appreciate that the terms “oligonucleotide” and “nucleic acid molecule” and the like may encompass a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. In certain embodiments, a polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

In some embodiments, a DNAzyme comprises a guanine-rich DNAzyme. In certain embodiments, the described guanine-rich DNAzyme a DNAzyme comprises guanine-rich extension(s) from either the 5′ and/or 3′ substrate-binding domain that is/are noncomplementary to the target sequence (“5′ overhang” and “3′ overhang”, respectively). In some embodiments, at least 30% of the nucleotides in the 5′ and/or 3′ overhang are guanine (G). In other embodiments, at least 30% of the nucleotides in both the 5′ and/or 3′ overhangs are guanine (G). In other embodiments, at least 35%, at least 40%, at least 45%, at least 50%, at least 55%, at least 60%, at least 65%, or at least 70%, at least 75%, at least 80%, at least 85%, at least 90%, at least 95%, or 100% of the nucleotides in the 5′ and/or 3′ overhang are guanine (G). In other embodiments, at least 30% of the nucleotides the combined sequences of both overhangs are guanine (G). In other embodiments, at least 35%, at least 40%, at least 45%, at least 50%, at least 55%, at least 60%, at least 65%, at least 70%, at least 75%, at least 80%, at least 85%, at least 90%, at least 95%, or 100% of the nucleotides in the combined sequences of both overhangs are guanine (G).

In some embodiments, nucleotides of the 5′ overhang are at least 35%, at least 40%, at least 45%, at least 50%, at least 55%, at least 60%, at least 65%, at least 70%, at least 75%, at least 80%, at least 85%, at least 90%, at least 95%, or at least 99% guanine (G). In some embodiments, nucleotides of the 3′ overhang are at least 30% guanine (G). In some embodiments, nucleotides of the 3′ overhang are at least 35%, at least 40%, at least 45%, at least 50%, at least 55%, at least 60%, at least 65%, at least 70%, at least 75%, at least 80%, at least 85%, at least 90%, at least 95%, or at least 99% guanine (G). In some embodiments, nucleotides of both the 5′ and 3′ overhangs are at least 30% guanine (G). In some embodiments, nucleotides of the 5′ and/or 3′ overhangs are at least 35%, at least 40%, at least 45%, at least 50%, at least 55%, at least 60%, at least 65%, at least 70%, at least 75%, at least 80%, at least 85%, at least 90%, at least 95%, or at least 99% guanine (G).

In some embodiments, provided herein are guanine-rich DNAzymes that bind to a RNA transcript and cleave the RNA transcript. In some embodiments, guanine-rich DNAzymes disclosed herein specifically bind to a RNA transcript. In some embodiments, guanine-rich DNAzymes disclosed herein specifically cleave a RNA transcript.

The term “binding” or “interacting” refers to an association, which may be a stable association, between two molecules, e.g., between a guanine-rich DNAzyme and RNA target. The association may be due to, for example but not limited to, electrostatic, hydrophobic, ionic, pi-stacking, coordinative, van der Waals, covalent and/or hydrogen-bond interactions under physiological conditions.

A skilled artisan would appreciate that “specific binding” may encompass the ability of a DNAzyme to bind to a predetermined RNA target. Typically, a DNAzyme specifically binds to its target with an affinity corresponding to a KD of about 10⁻⁷ M or less, about 10⁻⁸ M or less, or about 10⁻⁹ M or less and binds to the target with a KD that is significantly less (e.g., at least 2 fold less, at least 5 fold less, at least 10 fold less, at least 50 fold less, at least 100 fold less, at least 500 fold less, or at least 1000 fold less) than its affinity for binding to a non-specific and unrelated target (e.g., BSA, casein, or an unrelated cell, such as an HEK 293 cell or an E. coli cell).

In some embodiments, the guanine-rich DNAzymes provided herein comprise, in 5′ to 3′ order: (i) a 5′ arm comprising a sequence that base pairs with a first region of an RNA transcript; (ii) a DNAzyme catalytic core; and (iii) a 3′ arm comprising a sequence that base pairs with a second region of the RNA transcript positioned 5′ to the first region of the RNA transcript, wherein upon binding of the guanine-rich DNAzyme to the RNA transcript, the DNAzyme catalytic core cleaves the RNA transcript at a position between the first and second region of the RNA transcript, and wherein at least 30% of the nucleotides in the 5′ arm and/or the 3′ arm are guanine (G). In some embodiments, at least 30%, at least 35%, at least 40%, at least 45%, at least 50%, at least 55%, at least 60%, at least 65%, at least 70%, or at least 75% of the nucleotides in the 5′ arm and/or the 3′ arm are guanine (G). These embodiments may be freely combined with the embodiments of 5′ and 3′ overhang mentioned herein.

In some embodiments, the guanine-rich DNAzyme further comprises a 5′ overhang that does not base pair with the RNA transcript, comprising nucleotides immediately 5′ to the 5′ end of the first substrate binding domain/5′ arm, wherein at least 30% of the nucleotides in the 5′ extension are G. In other embodiments, at least 30%, at least 35%, at least 40%, at least 45%, at least 50%, at least 55%, at least 60%, at least 65%, at least 75%, at least 80%, at least 85%, at least 90%, at least 95%, or 100% of the nucleotides in the 5′ overhang are G.

In some embodiments, the guanine-rich DNAzyme further comprises a 3′ overhang that does not base pair with the RNA transcript, comprising nucleotides immediately 3′ to the 3′ end of the second substrate-binding domain/3′ arm, wherein at least 30% of the nucleotides in the 3′ extension are G. In other embodiments, at least 30%, at least 35%, at least 40%, at least 45%, at least 50%, at least 55%, at least 60%, at least 65%, at least 75%, at least 80%, at least 85%, at least 90%, at least 95%, or 100% of the nucleotides in the 3′ overhang are G.

In some embodiments, the guanine-rich DNAzyme further comprises both 5′ and 3′ overhangs, wherein at least 30% of the total nucleotides in the 5′ and 3′ overhangs are G. In other embodiments, at least 30%, at least 35%, at least 40%, at least 45%, at least 50%, at least 55%, at least 60%, at least 65%, at least 75%, at least 80%, at least 85%, at least 90%, at least 95%, or 100% of the total nucleotides in the 5′ and 3′ overhangs are G.

There are a number of different types of DNAzyme catalytic cores known in the art, some of which are listed in Table 1. A skilled artisan would recognize that a “DNAzyme” disclosed herein, may encompass a DNAzyme comprising any catalytic core, including but not limited to the types listed in Table 1. DNAzymes are often named based on their catalytic core sequences, for example but not limited to 10-23 DNAzymes, 8-17 DNAzymes, etc.

TABLE 1
Representative embodiments of DNAzyme catalytic
cores.

| DNAzymes | | SEQ |
|---|---|---|
| type | Catalytic Core Sequence | ID NO: |
| 10-23 | 5′ ggctagctacaacga 3′ | 1 |
| 8-17 | 5′ ccgagccggacga 3′ | 2 |
| E1111 | 5′ gtcagcgacacgaa 3′ | 3 |
| E2112 | 5′ gtcagtgactcgaa3′ | 4 |
| E5112 | 5′ gtcagctgactcgaa 4′ | 5 |
| bipartite | 5′ aggaggtaggggttccgctc 3′ | 6 |

In certain embodiments, the DNAzyme is a 10-23 class DNAzyme, e.g., as illustrated in FIG. 1.

The guanine-rich DNAzymes targeting a RNA transcript described herein may comprise any type of DNAzyme catalytic core, including but not limited to the types listed in Table 1. In some embodiments, the DNAzyme catalytic core is a 10-23 catalytic core, a 8-17 catalytic core, a E1111 catalytic core, a E2112 catalytic core, a E5112 catalytic core, or a bipartite catalytic core. In certain embodiments, the DNAzyme catalytic core comprises a 10-23 catalytic core. In other embodiments, the DNAzyme catalytic core comprises a 8-17 catalytic core, a E1111 catalytic core, a E2112 catalytic core, or a E5112 catalytic core In other embodiments, the DNAzyme catalytic core comprises a bipartite catalytic core.

In some embodiments, the DNAzyme catalytic core comprises the nucleic acid sequence selected from any one of SEQ ID NOs: 1-6. In some embodiments, the sequence of the DNAzyme catalytic core is that set forth in SEQ ID NO: 1. In other embodiments, the sequence of the DNAzyme catalytic core is that set forth in SEQ ID NO: 2. In other embodiments, the sequence of the DNAzyme catalytic core is that set forth in SEQ ID NO: 3, SEQ ID NO: 4, SEQ ID NO: 5, or SEQ ID NO: 6.

The guanine-rich DNAzymes targeting a RNA transcript described herein may bind to any region of an RNA transcript, including 5′ untranslated region, 3′ untranslated region, or the coding region.

In certain embodiments, the described DNAzyme binds to an RNA transcript through hybridization of the 5′ arm to the first region of the RNA transcript, and of the 3′ arm to the second region of the RNA transcript. The 5′ arm can be either fully complementary (100% complementary) to the first region of the RNA transcript; or partially complementary to the first region of the RNA transcript with no more than 1 mismatch; or in other embodiments no more than 2 mismatches. The 3′ arm can be either fully complementary (100% complementary) to the second region of the RNA transcript; or partially complementary to the second region of the RNA transcript with no more than 1 mismatch; or in other embodiments no more than 2 mismatches.

One skilled in the art would appreciate that two nucleic acid sequences “complement” one another or are “complementary” to one another if they base pair with one another at the indicated position(s).

In some embodiments, the total number of mismatches of the two substrate-binding domains to the first and second regions of the RNA transcript are no more than 1. In some embodiments, the total number of mismatches are no more than 2. In some embodiments, the total number of mismatches are no more than 3. In some embodiments, the total number of mismatches are no more than 4.

In some embodiments, the 5′ or 3′ arm may be 6-15 nucleotides (e.g., 6, 7, 8, 9, 10, 11, 12, 13, 14, or 15 nucleotides) in length. In some embodiments, the 5′ arm comprises 6-15 nucleotides. In some embodiments, the 5′ arm comprises 6, 7, 8, 9, 10, 11, 12, 13, 14, or 15 nucleotides. In some embodiments, the 3′ arm comprises 6-15 nucleotides. In some embodiments, the 3′ arm comprises 6, 7, 8, 9, 10, 11, 12, 13, 14, or 15 nucleotides. In other embodiments, the 3′ and 5′ arms are the same or different lengths. In some embodiments, the 5′ arm and the 3′ arm are each 6, 7, 8, 9, 10, 11, 12, 13, 14, or nucleotides in length. In some embodiments, the 5′ arm and the 3′ arm are each 9 nucleotides in length. In some embodiments, the 5′ arm and the 3′ arm comprise different lengths of nucleotides independently selected from 6, 7, 8, 9, 10, 11, 12, 13, 14, or 15 nucleotides. These embodiments may be freely combined with each other.

In other embodiments, the substrate binding domains can comprise RNA bases, DNA bases or combinations of RNA and DNA bases.

A skilled artisan would appreciate that the terms “nucleotide base”, “nucleotide”, “base”, and “nucleic acid base” may be used interchangeably having all the same qualities and meaning. In some embodiments, a base comprises a DNA or RNA base, or any modifications thereof.

In some embodiments, a DNA base comprises any of adenine (A), thymine (T), guanine (G), or cytosine (C), or a combination thereof. In some embodiments, the DNA base comprises a modified DNA base. In some embodiments, the modified DNA base comprises a 8-aza-7-deazaguanosine.

In other embodiments, a RNA base comprises any of adenine (A), uracil (U), guanine (G), or cytosine (C), or a combination thereof. In some embodiments, the RNA base comprises a modified RNA base.

In certain embodiments, modified bases (which may be, for example, non-naturally occurring bases) preserving the base pair specificity of the parent DNA or RNA base are considered equivalent to the DNA or RNA parent bases, e.g., a sequence mentioned herein as containing “guanine” contain instead modified forms of guanine preserving the base pair specificity of guanine.

In other embodiments, the DNAzyme comprises a 5′ extension and/or a 3′ extension from the 5′ and/or 3′ arm, respectively, wherein the extension does not interfere with the binding of the RNA transcript, or interference is minimal, wherein specific binding still occurs. In some embodiments, the DNAzyme comprises a 5′ extension and/or a 3′ extension from the 5′ and/or 3′ arm, respectively, wherein the extension does not interfere with the cleavage of the RNA transcript, or interference is minimal, wherein cleavage of the RNA transcript still occurs. In some embodiments, the DNAzyme comprises a 5′ and/or 3′ overhangs, wherein the overhangs do not interfere with the binding and cleavage of the RNA transcript, or interference is minimal, wherein specific binding and cleavage of the RNA transcript still occurs.

In some embodiments, a 5′ extension comprises 1 to 10 nucleotides in length. In other embodiments, the 5′ extension is 2-6 nucleotides in length. In other embodiments, a 5′ extension is 2, 3, 4, 5, 6, 7, 8, 9, or 10, etc. nucleotides in length. In other embodiments, the 5′ extension is 2-9 nucleotides in length. In other embodiments, the 5′ extension is 2-8, 2-6, 2-5, 2-4, or 2-3 nucleotides in length. In some embodiments, a 3′ extension is 1 to 10 nucleotides in length. In other embodiments, the 3′ extension is 2-9 nucleotides in length. In other embodiments, the 3′ extension is 2-8, 2-6, 2-5, 2-4, or 2-3 nucleotides in length. In some embodiments, the 3′ extension is 2, 3, 4, 5, 6, 7, 8, 9, or 10, etc. nucleotides in length. In some embodiments, a 3′ extension is 1-10 nucleotides in length, and the 5′ extension is 1-10 nucleotides in length. In other embodiments, the 5′ and 3′ extensions are both independently selected from 1-13 nucleotides in length. In other embodiments, the 5′ and 3′ extensions are both independently selected from 1-8, 1-6, 1-5, 1-4, 1-3, 2-13, 2-8, 2-6, 2-5, 2-4, or 2-3 nucleotides in length. In some embodiments, a 5′ extension is 1, 2, 3, 4, 5, 6, 7, 8, 9, 10, 11, 12, 13, etc. nucleotides in length and a 3′ extension is 1, 2, 3, 4, 5, 6, 7, 8, 9, 10, 11, 12, 13, etc. nucleotides in length, wherein the length of the extensions is the same. In some embodiments, a 5′ extension is 2, 3, 4, 5, 6, 7, 8, 9, or 10, etc. nucleotides in length and a 3′ extension is 2, 3, 4, 5, 6, 7, 8, 9, or 10, etc. nucleotides in length, and the length of the extensions is different.

Throughout this application, various embodiments may be presented in a range format. It should be understood that the description in range format is merely for convenience and brevity and should not be construed as an inflexible limitation on the scope of the DNAzymes and uses thereof, disclosed herein. Accordingly, the description of a range should be considered to have specifically disclosed all the possible subranges as well as individual numerical values within that range. For example, description of a range such as from 1 to 6 should be considered to have specifically disclosed subranges such as from 1-3, from 1-4, from 1-5, from 2-4, from 2-6, from 3-6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

The extensions may comprise RNA bases, DNA bases or combinations of RNA and DNA bases. The extensions may comprise naturally-occurring bases (A, C, Me-C, T, G, U, I, etc.), or artificial nucleotides (LNA, phosphorothioate, 2-O-F, 2-O-Methyl, 2-O-Methoxyethyl, etc.), or combinations of both.

In some embodiments, the 5′ arm, the 3′ arm, the 5′ overhang, and/or the 3′ overhang of the described DNAzymes comprise(s) at least one tandem repeat of G (2 or more consecutive Gs). In some embodiments, the 5′ arm comprises at least one tandem repeat of G. In some embodiments, the 3′ arm comprises at least one tandem repeat of G. In some embodiments, the 5′ arm and the 3′ arm each comprise at least 1 tandem repeat of G. In some embodiments, the 5′ extension comprises 2 or more consecutive Gs. In some embodiments, the 3′ extension comprises at least 1 tandem repeat of G. In some embodiments, the 5′ extension and the 3′ extension of the described DNAzyme both comprise at least 1 tandem repeat of G. In some embodiments, each of the 5′ arm, the 3′ arm, the 5′ extension, and the 3′ extension of the guanine-rich DNAzyme described herein comprise at least 1 tandem repeat of G.

In certain embodiments, a tandem repeat comprises 2 consecutive Gs. In some embodiments, a tandem repeat comprises at least 2 consecutive Gs. In some embodiments, any tandem repeat of G described herein contains at least 3 consecutive Gs. In still other embodiments, any tandem repeat of G described herein contains at least 4 consecutive Gs. In yet other embodiments, any tandem repeat of G described herein contains 2-3 consecutive Gs; 2-4 consecutive Gs; or 3-4 consecutive Gs. In some embodiments, the 5′ and 3′ arms each comprise at least 1 tandem repeat consisting of 2 consecutive Gs. In some embodiments, the 5′ arm comprises at least 1 tandem repeat consisting of 2 consecutive Gs. In some embodiments, the 3′ arm comprises at least 1 tandem repeat consisting of 2 consecutive Gs.

In some embodiments, the 5′ arm, the 3′ arm, the 5′ overhang, and/or the 3′ overhang of the guanine-rich DNAzyme described herein comprises at least 1 guanine (G) at every interval of 3 nucleotides.

In certain embodiments, the described DNAzyme forms a structure, which may, in more specific embodiments, be any structure mentioned herein.

In some embodiments, the 5′ arm or the 3′ arm of the guanine-rich DNAzyme described herein forms a structure within the domain itself (e.g., within the 5′ arm or within the 3′ arm). In some embodiments, the 5′ arm of the guanine-rich DNAzyme described herein forms a structure with the 3′ arm.

In some embodiments, the 5′ overhang or the 3′ overhang of the guanine-rich DNAzyme described herein forms a structure within the overhang itself (e.g., within the 5′ extension or within the 3′ extension). In some embodiments, the 5′ overhang of a described guanine-rich DNAzyme forms a structure with the 3′ overhang.

In some embodiments, the 5′ arm of the guanine-rich DNAzyme described herein forms a structure with the 5′ overhang or the 3′ overhang. In some embodiments, the 3′ arm of the guanine-rich DNAzyme described herein forms a structure with the 5′ overhang or the 3′ overhang.

In some embodiments, the structure formed does not interfere with the binding or the cleavage of the RNA transcript. In some embodiments, the structure is a structure formed between DNA strands based on Hoogsteen or wobble base pairing. In certain embodiments, the structure is a G-quadruplex, G-triplex, or H-DNA. In some embodiments, the 5′ arm and the 3′ arm of the guanine-rich DNAzyme described herein form a G-quadruplex structure. In some embodiments, a 5′ and/or 3′ overhang is/are part of a G-quadruplex structure formed. Methods for predicting secondary structures of nucleotides are known in the art, and are described, for example, in Kikin et al. (2006) QGRS Mapper: a web-based server for predicting G-quadruplexes in nucleotide sequences Nucleic Acids Research 2006 July; 34 (Web Server issue):W676-W682; Brázda et al (2019) G4Hunter web application: a web server for G-quadruplex prediction, Bioinformatics, Volume 35, Issue 18, 15 Sep. 2019, pages 3493-3495; and Buske et al (2012) Triplexator: detecting nucleic acid triple helices in genomic and transcriptomic data. Genome Res. 2012 July; 22(7):1372-81.

In some embodiment, the structure is an intramolecular structure or an intermolecular structure formed by up to 4 nucleotides.

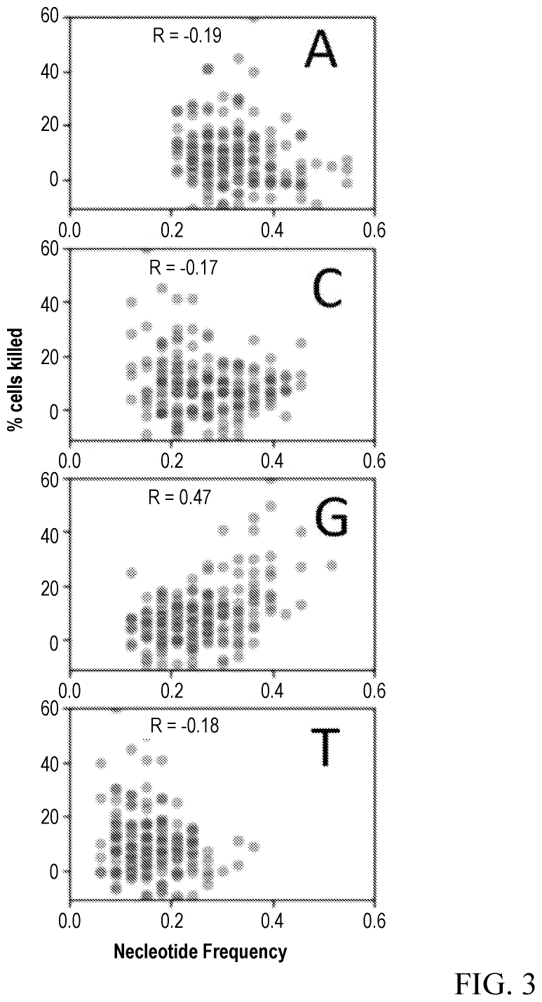

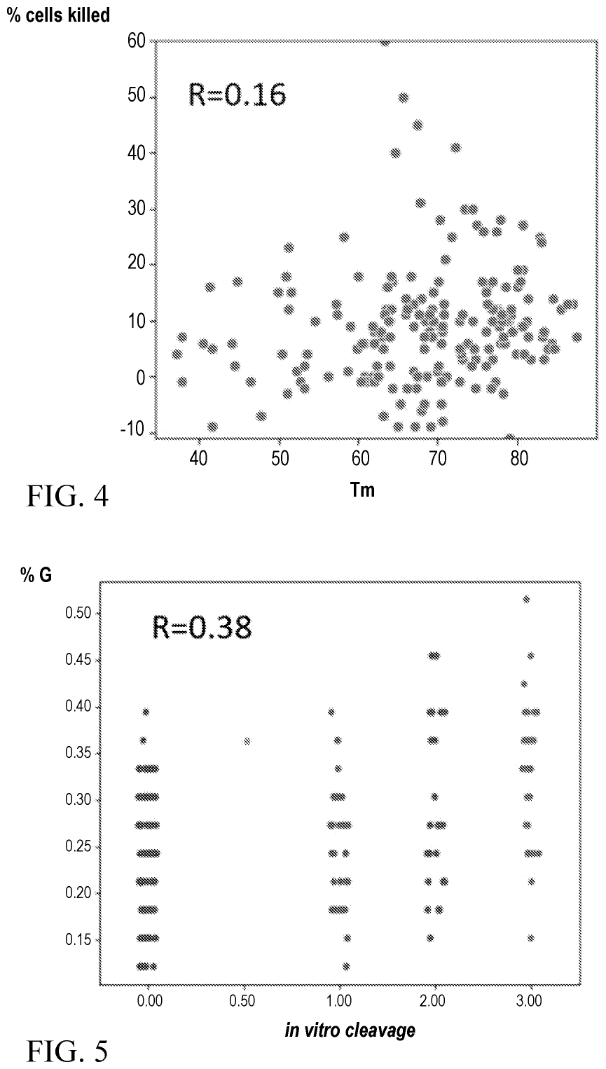

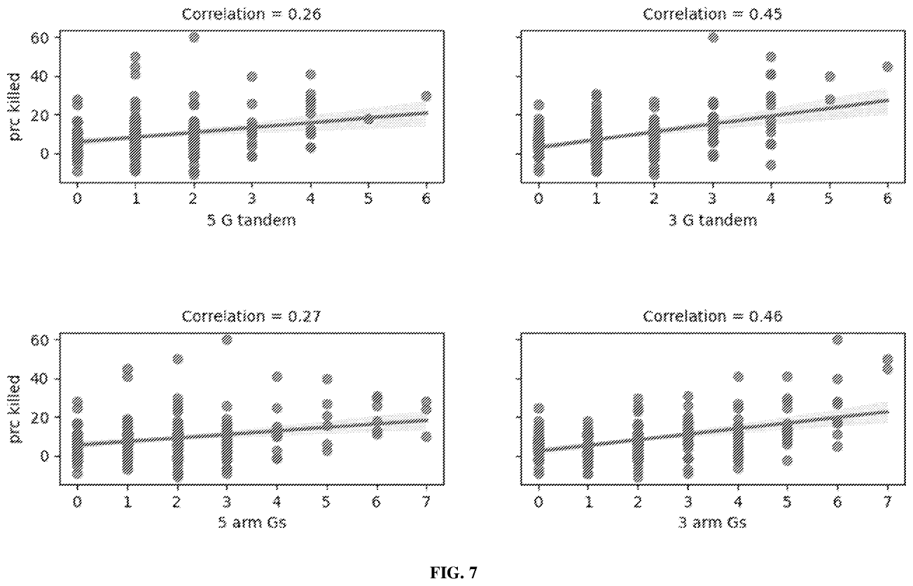

In some embodiments, the guanine-rich DNAzymes provided herein are able to cleave a RNA transcript. In some embodiments, the guanine-rich DNAzymes provided herein are able to reduce expression level (e.g., mRNA level and/or protein level) of a gene. In some embodiments, the guanine-rich DNAzymes, for example but not limited to the herein-described DNAzymes targeting p21, USP7, KRAS, or BIRC5 mRNA, are able to inhibit cell growth and/or replication, and/or to induce cell death in vitro (see, for example FIG. 3, FIG. 4, FIG. 6). In other embodiments, the described guanine-rich DNAzymes are able to inhibit cell growth and/or replication, and/or to induce cell death in vivo.

In some embodiments, the RNA transcript is from a prokaryotic gene (e.g., a bacterial gene). In some embodiments, the RNA transcript is from a eukaryotic gene (e.g., a human gene). In some embodiments, the RNA transcript is a messenger RNA (mRNA) or a pre-messenger RNA (pre-mRNA) of a eukaryotic gene. In specific embodiments, the RNA transcript is a p21 messenger RNA (mRNA), and the guanine-rich DNAzyme targets the p21 mRNA. In other embodiments, the described DNAzyme targets an mRNA selected from USP7, KRAS, and BIRC5.

P21 gene encodes a potent cyclin-dependent kinase inhibitor. The encoded p21 protein binds to and inhibits the activity of cyclin-cyclin-dependent kinase (cdk) 2 or -cdk 4 complexes, and thus functions as a regulator of cell cycle progression at G1. The expression of P21 gene is tightly controlled by the tumor suppressor protein p53, through which this protein mediates the p53-dependent cell cycle G1 phase arrest in response to a variety of stress stimuli. P21 protein can interact with proliferating cell nuclear antigen, a DNA polymerase accessory factor, and plays a regulatory role in S phase DNA replication and DNA damage repair. It is specifically cleaved by CASP3-like caspases, which thus leads to a dramatic activation of cyclin-dependent kinase 2, and may be instrumental in the execution of apoptosis following caspase activation.

In some embodiments, the 5′ arm of the guanine-rich DNAzyme targeting the P21 mRNA comprises a nucleic acid sequence 5′-GGGAAAGGA-3′ (SEQ ID NO: 7), and the 3′ arm comprises a nucleic acid sequence 5′-AAGGGGGAG-3′ (SEQ ID NO: 8). In all substrate-binding domain sequences disclosed herein, where a thymidine base is identified, a uracil base is also contemplated.

The pairs of the substrate-binding domains described above can be combined with any catalytic core sequence described herein to form guanine-rich DNAzymes that targets the P21 mRNA. Such P21-targeting guanine-rich DNAzymes may comprise a nucleic acid sequence selected from SEQ ID NOs: 9-14 (e.g., SEQ ID NO: 9) in Table 2 below. In certain embodiments, P21-targeting guanine-rich DNAzymes comprise a nucleic acid sequence selected from SEQ ID NOs: 10-14. In certain embodiments, P21-targeting guanine-rich DNAzymes comprise a nucleic acid sequence selected from SEQ ID NOs: 10-14 and 17-34. In certain embodiments, P21-targeting guanine-rich DNAzymes comprise a nucleic acid sequence selected from SEQ ID NOs: 17-34.

Table 2: Guanine-rich DNAzymes targeting P21 mRNA.

TABLE 2
Guanine-rich DNAzymes targeting P21 mRNA.

| Guanine-rich DNAzyme Sequences | SEQ ID NO: |
|---|---|
| 5′-GGGAAAGGA ggctagctacaacga AAGGGGGAG-3′ | 9 |
| 5′-GGGAAAGGA ccgagccggacga AAGGGGGAG-3′ | 10 |
| 5′-GGGAAAGGA gtcagcgacacgaa AAGGGGGAG-3′ | 11 |
| 5′-GGGAAAGGA gtcagtgactcgaa AAGGGGGAG-3′ | 12 |
| 5′-GGGAAAGGA gtcagctgactcgaa AAGGGGGAG-3′ | 13 |
| 5′-GGGAAAGGA aggaggtaggggttccgctc AAGGGGGAG-3′ | 14 |

In some embodiments, a 5′ arm or a 3′ arm comprises modifications. In some embodiments, modifications comprise substitution mutations. In some embodiments, the substation mutations comprise substituting in a guanine base in place of any of adenine, cytosine, or thymine. In some embodiments, a 5′ arm is modified wherein at least one guanine base is substituted in place of any other base. In some embodiments, a 5′ arm is modified wherein 2 guanine bases are substituted in place of any other 2 bases. In some embodiments, a 5′ arm is modified wherein 3 guanine bases are substituted in place of any other 3 bases. In some embodiments, a 3′ arm is modified wherein one guanine base is substituted in place of any other base. In some embodiments, a 3′ arm is modified wherein 2 guanine bases are substituted in place of any other 2 bases. In some embodiments, a 3′ arm is modified wherein 3 guanine bases are substituted in place of any other 3 bases. In some embodiments, the 5′ and 3′ arms are modified wherein one guanine base is substituted in place of any other base. In other embodiments, the 5′ and 3′ arms are modified, wherein 2 guanine bases are substituted in place of any other 3 bases. In other embodiments, the 5′ and 3′ arms are modified, wherein 3 guanine bases are substituted in place of any other 3 bases.

In some embodiments, the guanine-rich DNAzymes provided herein comprise one or more chemical modifications. In some embodiments, the one or more chemical modifications are selected from the group consisting of base modifications, sugar modifications, and inter-nucleotide linkage modifications. In some embodiments, the one or more chemical modifications are selected from the group consisting of locked nucleic acids (LNA), phosphorothioate, 2-O-fluoro, 2-O-methyl, 2-O-methoxyethyl, and methyl-Cytosine.

Non-limiting examples of modifications are provided in Table 3.

TABLE 3
Embodiments of chemical modifications.

| Terminal | Sugar ring | Nitrogen base | Backbone |
|---|---|---|---|
| biotin | 2′-OH | 5′-BzdU | Phosphorothioate |
| | (RNA) | | |
| Inverted-dT | 2′-OMe | Naphtyl | Methylphosphorothioate |
| PEG (0.5-40 kDa) | 2′-F | Triptamino | Phosphorodithioate |
| Cholesterol | 2′-NH2 | Isobutyl | Triazole |
| Albumin | LNA | 5-Methyl Cytosine | Amide (PNA) |
| Chitin (0.5-40 kDa) | UNA | Alkyne | Alkyne |
| | | (dibenzocyclooctyne) | (dibenzocyclooctyne) |
| Chitosan (0.5-40 kDa) | 2′-F ANA | Azide | Azide |
| Cellulose (0.5-40 kDa) | L-DNA | Maleimide | Maleimide |
| Terminal amine | CeNA | | |
| (alkyne chain with | | | |
| amine) | | | |
| Alkyl | TNA | | |
| (dibenzocyclooctyne) | | | |
| Azide | HNA | | |
| Thiol | | | |
| Maleimide | | | |
| N-hydroxysuccinimide | | | |

In certain embodiments, the guanine-rich DNAzymes comprise a terminal modification. In some embodiments, the guanine-rich DNAzymes are chemically modified with poly-ethylene glycol (PEG). In some embodiments, the PEG comprises 0.5-40 kDa PEG. In some embodiment, the PEG is attached to the 5′ end of the DNAzyme.

In some embodiments, the guanine-rich DNAzymes comprise a 5′ end cap. In some embodiments, the 5′ end cap comprises an inverted thymidine, biotin, albumin, chitin, chitosan, cellulose, terminal amine, alkyne, azide, thiol, maleimide, or N-hydroxysuccinimide. In certain embodiments, the guanine-rich DNAzymes comprise a 3′ end cap. In some embodiments, the 3′ end cap comprises an inverted thymidine; or bases modified with biotin, albumin, chitin, chitosan, cellulose, terminal amine, alkyne, azide, thiol, maleimide, or N-hydroxysuccinimide.

In certain embodiments, the guanine-rich DNAzymes provided herein comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, or 50) modified sugars. In some embodiments, the guanine-rich DNAzymes comprise one or more 2′ sugar substitutions (e.g., a 2′-fluoro, a 2′-amino, or a 2′-O-methyl substitution). In certain embodiments, the guanine-rich DNAzymes comprise locked nucleic acid (LNA), unlocked nucleic acid (UNA) and/or 2′deozy-2′fluoro-D-arabinonucleic acid (2′-FANA) sugars in their backbone.

In certain embodiments, the guanine-rich DNAzymes comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, or 50) methylphosphonate internucleotide bonds and/or a phosphorothioate (PS) internucleotide bonds. In certain embodiments, the guanine-rich DNAzymes comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, or 50) triazole internucleotide bonds. In certain embodiments, the guanine-rich DNAzymes are modified with a cholesterol or a dialkyl lipid (e.g., on their 5′ end, 3′ end, or both ends).

In some embodiments, the guanine-rich DNAzymes comprise one or more modified bases (e.g., 5-(N-benzylcarboxyamide)-2′-deoxyuridine) [5-BzdU], β-naphthyl-, tryptamine, or Isobutyl substituted bases; 5-methyl cytosine, or bases modified with alkyne, dibenzocyclooctyne, azide, or maleimide).

In certain embodiments, the guanine-rich DNAzymes provided herein are DNA DNAzymes (e.g., D-DNA DNAzymes or enantiomer L-DNA DNAzymes). In other embodiments, the guanine-rich DNAzymes provided herein are RNA DNAzymes (e.g., D-RNA DNAzymes or enantiomer L-RNA DNAzymes). In other embodiments, the guanine-rich DNAzymes comprise a mixture of DNA and RNA.

In certain embodiments, the guanine-rich DNAzymes provided herein are linked to a penetration-enhancing moiety. The cell penetration-enhancing moiety may comprise an aptamer, a small molecule, a polypeptide, a nucleic acid, a protein, or an antibody. In some embodiments, the guanine-rich DNAzyme is covalently linked to the penetration-enhancing moiety. In some embodiments, the guanine-rich DNAzyme is non-covalently linked to the penetration-enhancing moiety. In some embodiments, the guanine-rich DNAzyme is directly linked to the penetration-enhancing moiety. In some embodiments, the guanine-rich DNAzyme is linked to the penetration-enhancing moiety via a linker.

As used herein, the term “penetration-enhancing moiety” encompasses any moiety known in the art to facilitate actively or passively, or to enhance penetration of compounds into the cells. In some embodiments, a penetration-enhancing moiety encompasses any moiety that facilitates actively or passively, or enhances permeability of a guanine-rich DNAzyme into the cells.

In some embodiments, the penetration-enhancing moiety comprises a polysaccharide, a synthetic nucleoside base, an inverted nucleoside base, cholesterol, other sterols, lipids, membrane lipids, or synthetic lipids. In some embodiments, a penetration-enhancing moiety comprises cholesterol. In some embodiments, a penetration-enhancing moiety comprises a cell penetrating peptide (CPP). In some embodiments, a penetration-enhancing moiety comprises alpha-tocopherol. In some embodiments, a penetration-enhancing moiety comprises Vitamin B12.

In some embodiments, the cell penetration-enhancing moiety is cholesterol, linked directly or via a linker to the 5′ or 3′ terminus (or both) of the guanine-rich DNAzyme. In some embodiments, the cell penetration-enhancing moiety is cholesterol-TEM, wherein TEM is a 15-atom triethylene glycol spacer. In certain embodiments, the linker is an alkyl or alkoxy group, a non-limiting examples of which is cholesterol-TEG (triethylene glycol). In some embodiments, the chain length of the linker is between 5-20, in other embodiments between 5-16, in other embodiments between 10-16 atoms.

In some embodiments, the guanine-rich DNAzyme is encapsulated in a liposome, conjugated to a micro- or nano-particle, or embedded in a polymer matrix such as gel, PLGA, PEG, etc.

The guanine-rich DNAzymes may be synthesized by methods which are well known to the skilled person. For example, the guanine-rich DNAzymes may be chemically synthesized, e.g., on a solid support. Solid phase synthesis may use phosphoramidite chemistry. Briefly, the synthesis cycle starts with the removal of the acid-labile 5′-dimethoxytrityl protection group (DMT, “Trityl”) from the hydroxyl function of the terminal, support-bound nucleoside by UV-controlled treatment with an organic acid. The exposed highly-reactive hydroxyl group is then available to react in the coupling step with the next protected nucleoside phosphoramidite building block, forming a phosphite triester backbone. Next, the acid-labile phosphite triester backbone is oxidized to the stable pentavalent phosphate trimester. If a phosphorothioate modification is desired at a specific backbone position, the acid labile phosphite trimester backbone is sulfuridized at this step, instead of the oxidation process, to generate a P═S bond rather than a P═O. Successively, all the unreacted 5′-hydroxyl groups are acetylated (“capped”) in order to block these sites during the next coupling step, avoiding internal mismatch sequences. Following the capping step, the cycle starts again by removal of the DMT-protection group and successive coupling of the next base according to the desired sequence. Finally, the oligonucleotide is cleaved from the solid support and all protection groups are removed from the backbone and bases.

### Nucleic Acids/Vectors

In certain embodiments, disclosed herein is a nucleic acid comprising one or more guanine-rich DNAzymes described herein. In some embodiments, each guanine-rich DNAzyme sequence is operably linked to an origin of replication and to a termination site.

One skilled in the art would appreciate that the term “operably linked” encompasses an arrangement of elements that allows them to be functionally related.

In some embodiments, each guanine-rich DNAzyme sequence is operably linked to an origin of replication and to a termination site such that each guanine-rich DNAzyme is separately replicated by the DNA replication machinery.

In other embodiments, the whole nucleic acid comprising one or more guanine-rich DNAzymes is operably linked to an origin of replication and to a termination site, wherein the nucleic acid comprises a cleavable sequence between each guanine-rich DNAzyme sequence. The cleavable sequence may be a hairpin-forming sequence, e.g., an enzymatically cleavable hairpin. In some embodiments, the nucleic acid comprising one or more guanine-rich DNAzymes is replicated by the DNA replication machinery and consequently spliced or parsed either via self-splicing or via enzymatic splicing to produce one or more guanine-rich DNA DNAzymes.

In certain embodiments, disclosed herein is a nucleic acid comprising complementary sequences of one or more guanine-rich DNAzymes described herein.

In some embodiments, the complementary sequence of each guanine-rich DNAzyme is operably linked to a promoter. In some embodiments, the complementary sequence of each guanine-rich DNAzyme is transcribed to produce a guanine-rich RNA DNAzyme.

In other embodiments, the whole nucleic acid comprising complementary sequences of one or more guanine-rich DNAzymes is operably linked to a promoter, wherein the nucleic acid encodes a cleavable sequence between the complementary sequence of each guanine-rich DNAzyme. In some embodiments, the cleavable sequence is a hairpin-forming sequence, e.g., an enzymatically cleavable hairpin. In some embodiments, the nucleic acid comprising complementary sequences of one or more guanine-rich DNAzymes is transcribed by the transcription machinery and consequently spliced or parsed either via self-splicing or via enzymatic splicing to obtain one or more guanine-rich RNA DNAzymes.

In certain embodiment, disclosed herein is a vector comprising a nucleic acid described herein.

A skilled artisan would appreciate that the terms “vector” and “expression vector” may be used interchangeably, having the same qualities and meanings. In some embodiments, a vector comprises any viral or non-viral vector such as plasmid, virus, retrovirus, bacteriophage, cosmid, artificial chromosome (bacterial or yeast), phage, binary vector in double or single stranded linear or circular form, or nucleic acid sequence that is able to transform host cells and optionally capable of replicating in a host cell. The vector may contain an optional marker suitable for use in the identification of transformed cells, e.g., tetracycline resistance or ampicillin resistance. A cloning vector may or may not possess the features necessary for it to operate as an expression vector.

In one embodiment, the vector is a plasmid. In another embodiment, the vector is a phage.

In some embodiments, the guanine-rich DNAzymes described herein are obtained upon replication or transcription of a vector described herein.

In some embodiments, the vectors described herein are conjugated to a penetration-enhancing moiety.

### Pharmaceutical Compositions

In certain embodiments, provided herein are pharmaceutical compositions comprising a guanine-rich DNAzyme. In some embodiments, a pharmaceutical composition comprises a therapeutically effective amount of a guanine-rich DNAzyme.

A skilled artisan would recognize that an “effective amount” (or, “therapeutically effective amount”) may encompass an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease, for example but not limited to cancer, neoplasms, other diseases resulting from accumulation of senescent cells, bacterial infections, immune diseases, and viral diseases. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the antigen-binding fragment administered.

In certain embodiments, provided herein are pharmaceutical compositions comprising a nucleic acid or a vector that comprises or encodes a guanine-rich DNAzyme. In some embodiments, provided herein are pharmaceutical compositions comprising a therapeutically effective amount of a nucleic acid or a vector comprising or encoding a guanine-rich DNAzyme. In some embodiments, the pharmaceutical compositions provided herein further comprise a pharmaceutically acceptable carrier.

In some embodiments, the pharmaceutical composition comprises a plurality of guanine-rich DNAzymes described herein. In some embodiments, the pharmaceutical composition comprises 2, 3, 4, 5, 6, 7, 8, 9, 10, 20, or more) guanine-rich DNAzymes described herein. In some embodiments, the pharmaceutical composition comprising a plurality of guanine-rich DNAzymes, comprises equal amounts of different guanine-rich DNAzymes. In some embodiments, the pharmaceutical composition comprises a plurality of guanine-rich DNAzymes at varying ratios.

Formulation of the pharmaceutical composition may be adjusted according to applications. In particular, the pharmaceutical composition may be formulated using a method known in the art to provide rapid, continuous, or delayed release of the active ingredient after administration to mammals.

In some embodiments, the pharmaceutical composition is in a form selected from the group consisting of tablets, pills, capsules, pellets, granules, powders, lozenges, sachets, cachets, elixirs, suspensions, dispersions, emulsions, solutions, infusions, syrups, aerosols, ophthalmic ointments, ointments, soft and hard gelatin capsules, suppositories, sterile injectable solutions, and sterile packaged powders.

In some embodiments, the pharmaceutical composition is suitable for administration via a route selected from the group consisting of oral, rectal, intramuscular, subcutaneous, intravenous, intraperitoneal, inhaled, intranasal, intraarterial, intravesical, intraocular, transdermal and topical.

The composition for oral administration may be in a form of tablets, troches, lozenges, aqueous, or oily suspensions, dispersible powders or granules, emulsions, hard or soft capsules, or syrups or elixirs. Pharmaceutical compositions intended for oral use may be prepared according to any method known to the art for the manufacture of pharmaceutical compositions and may further comprise one or more agents selected from sweetening agents, flavoring agents, coloring agents and preserving agents in order to provide pharmaceutically elegant and palatable preparations. Tablets contain the active agent in admixture with non-toxic pharmaceutically acceptable excipients, which are suitable for the manufacture of tablets. These excipients may be, e.g., inert diluents such as calcium carbonate, sodium carbonate, lactose, calcium phosphate, or sodium phosphate; granulating and disintegrating agents, e.g., corn starch or alginic acid; binders; and lubricating agents. The tablets may be coated utilizing known techniques to delay disintegration and absorption in the gastrointestinal tract and thereby provide an extended release of the drug over a longer period.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The compositions may contain other active compounds providing supplemental, additional, or enhanced therapeutic functions. Solid carriers or excipients are, for example, lactose, starch or talcum or liquid carriers such as, for example, water, fatty oils or liquid paraffins.

Other carriers or excipients which may be used include, but are not limited to, materials derived from animal or vegetable proteins, such as the gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; polypeptide/protein or polysaccharide complexes such as gelatin-acacia complexes; sugars such as mannitol, dextrose, galactose and trehalose; cyclic sugars such as cyclodextrin; inorganic salts such as sodium phosphate, sodium chloride and aluminium silicates; and amino acids having from 2-12 carbon atoms and derivatives thereof such as, but not limited to, glycine, L-alanine, L-aspartic acid, L-glutamic acid, L-hydroxyproline, L-isoleucine, L-leucine and L-phenylalanine.

Solutions or suspensions used for parenteral, intradermal, or subcutaneous application typically include the following components: a sterile diluent such as water for injection, saline solution, fixed oils, polyethylene glycols, glycerine, propylene glycol (or other synthetic solvents), antibacterial agents (e.g., benzyl alcohol, methyl parabens), antioxidants (e.g., ascorbic acid, sodium bisulfite), chelating agents (e.g., ethylenediaminetetraacetic acid), buffers (e.g., acetates, citrates, phosphates), and agents that adjust tonicity (e.g., sodium chloride, dextrose). The pH can be adjusted with acids or bases, such as hydrochloric acid or sodium hydroxide, for example. The parenteral preparation can be enclosed in ampules, disposable syringes or multiple dose glass or plastic vials.

Pharmaceutical compositions adapted for parenteral administration include, but are not limited to, aqueous and non-aqueous sterile injectable solutions or suspensions, which can contain antioxidants, buffers, bacteriostats and solutes that render the compositions substantially isotonic with the blood of an intended recipient. Such compositions can also comprise water, alcohols, polyols, glycerine and vegetable oils, for example. Extemporaneous injection solutions and suspensions can be prepared from sterile powders, granules and tablets. Such compositions may comprise a therapeutically effective amount of a guanine-rich DNAzyme and/or other therapeutic agent(s), together with a suitable amount of carrier to provide the form for proper administration to the subject.

The terms “pharmaceutically acceptable” and “pharmacologically acceptable” include molecular entities and compositions that do not produce an adverse, allergic, or other untoward reactions when administered to an animal, or human, as appropriate.

In some embodiments, the pharmaceutical composition is formulated to enhance the penetration of guanine-rich DNAzymes, nucleic acids, or vectors described herein into cells (e.g., prokaryotic cells or eukaryotic cells).

### Libraries of Guanine-Rich DNAzymes and Methods of Screening

In some embodiments, provided herein are libraries of DNAzymes, wherein at least 30% of the DNAzymes in the library are guanine-rich DNAzymes, as described herein. In some embodiments, provided herein are libraries of DNAzymes, wherein at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of the DNAzymes in the library are guanine-rich DNAzymes. In certain embodiments, all of the DNAzymes in a library of DNAzymes are guanine-rich DNAzymes described herein.

Those skilled in the art will appreciate, in light of the present disclosure, that guanine-rich DNAzyme libraries can be readily produced by selecting from a given DNAzyme library those sequences that are guanine-enriched in the arm sequences. In certain embodiments, a guanine-rich DNAzyme library is produced by first generating in silico an overall library of DNAzyme sequences meeting appropriate criteria, and then selecting those with guanine-rich arms. A non-limiting example of an overall DNAzyme sequence library, for exemplification only, is a given catalytic loop sequence paired with all possible arm sequences (in the case of both arms having 9 nucleotides each, the number of DNAzymes would be 4¹⁸, or approximately 68.7 billion sequences). Other non-limiting examples of overall DNAzyme sequence libraries, for exemplification only, are (a) one, or in other embodiments more than one, known catalytic loop sequence(s), or in other embodiments any possible loop sequence; wherein the loop sequence(s) is/are paired in with (b) a library of arm sequences complementary to a known genome, or in other embodiments a known transcriptome, or in other embodiments a known unspliced or spliced transcript, or in other embodiments all known transcripts of a particular gene, or in other embodiments one or more “hotspots” of a transcript that are believed to be amenable to DNAzyme attack. In still other embodiments, the target RNA, transcriptome, or genome is a target or group of targets whose susceptibility to DNAzymes was not previously characterized. The aforementioned embodiments of (a) loop sequences and (b) arm sequences may be freely combined.

Selection criteria for DNAzyme sequences with a guanine-rich 5′ arm, 3′ arm, or both arms may be any appropriate embodiment of guanine rich DNAzymes mentioned herein, each of which can be considered a separate embodiment. In certain embodiments, a computerized algorithm is used to generate the described library by selecting in silico guanine-rich sequences from an overall DNAzyme sequence library.

In still other embodiments, guanine-rich DNAzyme libraries are produced by selecting from an overall DNAzyme sequence library those sequences that have guanine-rich overhangs, for example as described hereinabove.

Selection criteria for DNAzyme sequences with a guanine-rich 5′ overhang, 3′ overhang, or both arms may be any appropriate embodiment of guanine rich DNAzymes mentioned herein, each of which can be considered a separate embodiment. In certain embodiments, a computerized algorithm is used to generate the described library by selecting in silico guanine-rich sequences from an overall DNAzyme sequence library.

In yet other embodiments, guanine-rich DNAzyme libraries are produced by selecting from an overall DNAzyme sequence library those sequences that have both guanine-rich arm sequences and guanine-rich overhangs, for example as described hereinabove.

In other embodiments, there is provided a computerized method of generating in silico a guanine-rich DNAzyme library, comprising the steps of: (a) obtaining a library of DNAzyme sequences meeting appropriate criteria, referred to as an “overall DNAzyme sequence library”; and (b) using a computerized algorithm to select from the overall DNAzyme sequence library those sequences that are guanine-enriched in the arm sequences; or in other embodiments having and guanine-rich overhangs; or in other embodiments having both guanine-rich arm sequences and guanine-rich overhangs, thereby generating a guanine-rich DNAzyme library. In some embodiments, the overall DNAzyme sequence library is provided in computer-readable format to a processor, and the analyzed sequence information is stored in an electronic storage location. In related embodiments, the processor is capable of performing at least 10⁸, 3×10⁸, 10⁹, 3×10⁹, 10¹⁰, 3×10¹⁰, 10¹¹, or 3×10¹¹ operations per second. In related embodiments, the DNAzyme sequence library is synthesized, after its sequences have been generated.

In other embodiments, there is provided a computer system (also “system” herein) programmed or otherwise configured for generating in silico a guanine-rich DNAzyme library. The system includes a central processing unit (CPU, also “processor” and “computer processor” herein), which can be a single core or multi core processor, or a plurality of processors for parallel processing. The system also includes memory (e.g., random-access memory, read-only memory, flash memory), an optional electronic storage unit 115 (e.g., hard disk), and a communications interface for communicating with one or more other users or systems. The communications interface can be configured to receive an overall DNAzyme sequence library, provided in computer-readable format, and also to transmit the sequences of a guanine-rich DNAzyme library generated by any of the described methods. The system optionally further comprises peripheral devices, such as cache, other memory, data storage and/or electronic display adapters. In related embodiments, each CPU is capable of performing at least 10⁸, 3×10⁸, 10⁹, 3×10⁹, 10¹⁰, 3×10¹⁰, 10¹¹, or 3×10¹¹ operations per second. In related embodiments, the DNAzyme sequence library is synthesized, after its sequences have been generated.

In some embodiments, the libraries of DNAzymes described herein comprise total 10²-10¹⁵ (e.g., 10², 10³, 10⁴, 10⁵, 10⁶, 10⁷, 10⁸, 10⁹, 10¹⁰, 10¹¹, 10¹², 10¹³, 10¹⁴, or 10¹⁵) unique DNAzymes. Those skilled in the art will appreciate that the maximum size of the library is governed by the number of sequence permutations that meet the requirements for inclusion in the library.

In some embodiments, a computer processor is used to select guanine-rich sequences from a large number of total DNAzymes in a computerized library. In some embodiments, a high-throughput processor is utilized. In some embodiments, the processor enables generation of a library of guanine-rich sequences from more than 10⁶, 10⁷, 10⁸, 10⁹, 10¹⁰, 10¹¹, 10¹², 10¹³, 10¹⁴, or 10¹⁵ total DNAzymes. In related embodiments, the processor is capable of performing at least 10⁸, 3×10⁸, 10⁹, 3×10⁹, 10¹⁰, 3×10¹⁰, 10¹¹, or 3×10¹¹ operations per second. In related embodiments, the DNAzyme sequence library is synthesized, after its sequences have been generated.

In certain embodiments, the described libraries enhance the efficiency of DNAzyme selection, by enabling identification of DNAzymes with higher potency, per a given library size. In other embodiments, the libraries decrease the cost and time necessary for successful identification of effective DNAzymes.

In certain embodiments, provided herein are methods of screening for a DNAzyme that cleaves a RNA transcript, comprising: (1) providing a library of DNAzymes described herein; (2) incubating the library of DNAzymes with the RNA transcript; (3) detecting the cleavage of the RNA transcript by one or more DNAzymes from the library.

In other embodiments, there is provided a method of screening for a DNAzyme with cytotoxic activity, comprising: (1) providing a library of DNAzymes described herein; (2) incubating the library of DNAzymes with a desired cell type; (3) detecting cytotoxicity in the cell type by one or more DNAzymes from the library. In further embodiments, the DNAzyme library is designed to recognize and cleave a desired target RNA expressed by the cell type. In more specific embodiments, the target RNA may be any of the RNAs or classes of RNA described herein. In certain embodiments, cleavage of the target RNA is also detected.

In some embodiments, the described screening method is a cell-free assay. In other embodiments, the method is a cell-based assay. In further embodiments, the incubation step (2) occurs in vitro, in vivo, or ex vivo. The RNA cleavage can be detected by any methods, for example, by gel electrophoresis, PCR, probe hybridization, sequencing, etc. In some embodiments, the methods further comprises detecting the expression level (e.g., mRNA level or protein level) or activity of the gene that corresponds to the RNA transcript.

### Therapeutic Methods and Other Uses

In some embodiments, provided herein are methods of cleaving a RNA transcript, comprising contacting the RNA transcript with a guanine-rich DNAzyme described herein.

In some embodiments, provided herein are methods of inhibiting expression of a gene, comprising contacting the RNA transcript with a guanine-rich DNAzyme described herein. In some embodiments, where an intact cell is the target, a cell containing the RNA transcript is contacted with the DNAzyme. In further embodiments, the DNAzyme is conjugated to a moiety that enables cell penetration. In still other embodiments, an RNA transcript is contacted with the described DNAzyme in vitro. In some embodiments, a method of inhibiting expression of a gene comprises reducing the mRNA level of the gene, reducing the level of the encoded protein, or reducing the levels both mRNA and it encoded protein. In some embodiments, methods of inhibiting expression of a gene result in increased cytotoxicity of the cell comprising said gene.

In some embodiments, provided herein are methods of treating a disease in a subject in need, said method comprising administering a guanine-rich DNAzyme described herein, wherein said guanine-rich DNAzyme targets a gene associated with said disease. In some embodiments, the disease is cancer. In other embodiments, the DNAzyme is administered to a subject in need thereof to reduce the number of senescent cells.

In other embodiments, a described DNAzyme is administered to treat a disease associated with, or caused at least in part by, expression of a particular transcript. In the case of cancer, for example, the DNAzyme is used to target an oncogene that aids in survival or proliferation of the cancer cells.

Non-limiting examples of cancers that can be targeted are carcinomas. The term “carcinoma” refers to malignancies of epithelial or endocrine tissues, including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., including malignant tumors composed of carcinomatous and sarcomatous tissues. An “adenocarcinoma” refers to a carcinoma derived from glandular tissue or in which the tumor cells form recognizable glandular structures.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CIVIL); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to, non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

In other embodiments, the described DNAzymes are used to treat bacterial infections. In more specific embodiments, the DNAzyme targets a bacterial antibiotic resistance gene (a gene conferring antibiotic resistance to a bacterium). Non-limiting examples of such genes are extended-spectrum beta-lactamases (ESBLs), penicillinases (EC: 3.5.2.6), cephalosporinases (EC: 3.5.2.6), and carbapenemases (EC: 3.5.2.6). Further embodiments of antibiotic resistance genes are described in Int. Patent. Appln. Pub. No. WO2022/097157A2 to Ido Bachelet et al., the contents of which are incorporated herein by reference.

In certain embodiments, the bacterium is a gram-positive bacterium, non-limiting examples of which are Actinomyces israelli, Bacillus species, Bacillus antracis, Brevibacillus, Clostridium, Clostridium perfringens, Clostridium tetani, Cornyebacterium, Corynebacterium diphtheriae, Enterococcus (e.g. Enterococcus faecium), Erysipelothrix rhusiopathiae, Lactobacillus, Listeria, Mycobacterium, Staphylococcus (e.g. Staphylococcus aureus), Streptomyces and Streptococcus.

In certain embodiments, the bacterium is a gram-negative non-limiting examples of which are Aerobacter, Aeromonas, Acinetobacter (e.g. Acinetobacter baumannii), Agrobacterium, Bacteroides, Bartonella, Bordetella, Borrelia, Brucella, Burkholderia, Campylobacter, Calymmatobacterium, Campylobacter, Capnocytophaga, Cardiobacterium, Citrobacter, Chlamydia, Chlamydophila, Eikenella, Enterobacter, Enterobacter aerogenes, Escherichia, Flavobacterium, Francisella, Fusobacterium, Fusobacterium nucleatum, Gardnerella, Haemophilus, Hafnia, Helicobacter, Kingella, Klebsiella (e.g. Klebsiella pneumoniae), Legionella, Leptospira, Morganella, Moraxella, Mycoplasma, Neisseria, Pasteurella (e.g. Pasteurella multocida), Plesiomonas, Prevotella, Proteus, Providencia, Pseudomonas (e.g. Pseudomonas aeruginosa), Porphyromonas, Rickettsia, Salmonella, Serratia, Shigella, Stenotrophomonas, Streptobacillus, Streptobacillus moniliformis, Stenotrophomonas, Spirillum, Treponema (e.g. Treponema pallidium, Treponema pertenue), Xanthomonas, Veillonella, Vibrio, and Yersinia.

In other embodiments, the described DNAzymes are used to treat a subject wish a disease or disorder caused by bacteria, non-limiting examples of which are actinomycosis, anaplasmosis, anthrax, bacillary angiomatosis, actinomycetoma, bacterial pneumonia, bacterial vaginosis, bacterial endocarditis, bartonellosis, botulism, boutenneuse fever, brucellosis, bejel, brucellosis spondylitis, bubonic plague, Buruli ulcer, Bairnsdale ulcer, bacillary dysentery, campylobacteriosis, Carrion's disease, cat-scratch disease, cellulitis, chancroid, chlamydia, chlamydia conjunctivitis, clostridial myonecrosis, cholera, Clostridium difficile colitis, diphtheria, Daintree ulcer, donavanosis, dysentery, erhlichiosis, epidemic typhus, fried rice syndrome, five-day fever, floppy baby syndrome, Far East scarlet-like fever, gas gangrene, glanders, gonorrhea, granuloma inguinale, human necrobacillosis, hemolytic-uremic syndrome, human ewingii ehrlichiosis, human monocytic ehrlichiosis, human granulocytic anaplasmosis, infant botulism, Izumi fever, Kawasaki disease, Kumusi ulder, lymphogranuloma venereum, Lemierre's syndrome, Legionellosis, leprosy, leptospirosis, listeriosis, Lyme disease, lymphogranuloma venereum, Malta fever, Mediterranean fever, myonecrosis, mycoburuli ulcer, mucocutaneous lymph node syndrome, meliodosis, meningococcal disease, murine typhus, Mycoplasma pneumonia, mycetoma, neonatal conjunctivitis, nocardiosis, Oroya fever, ophthalmia neonatorum, ornithosis, Pontiac fever, peliosis hepatis, pneumonic plague, postanginal shock including sepsis, pasteurellosis, pelvic inflammatory disease, pertussis, plague, pneumococcal infection, pneumonia, psittacosis, parrot fever, pseudotuberculosis, Q fever, quintan fever, rabbit fever, relapsing fever, rickettsialpox, Rocky Mountain spotted fever, rat-bite fever, Reiter syndrome, rheumatic fever, salmonellosis, scarlet fever, sepsis, septicemic plague, Searls ulcer, shigellosis, soft chancre, syphilis, streptobacillary fever, scrub typhus, Taiwan acute respiratory agent, Trench fever, trachoma, tuberculosis, tularemia, typhoid fever, typhus, tetanus, toxic shock syndrome, undulant fever, ulcus molle, Vibrio parahaemolyticus enteritis, Whitmore's disease, walking pneumonia, Waterhouse-Friderichsen syndrome, yaws, and yersiniosis.

In other embodiments, the described compositions are used to treat immune disorders, in particular those associated with overexpression of a gene or expression of a mutant gene. Examples of hematopoietic disorders or diseases include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy, e.g., atopic allergy.

In other embodiments, the described DNAzymes are used to treat viral diseases, including but not limited to hepatitis B, hepatitis C, herpes simplex virus (HSV), HIV-AIDS, poliovirus, and smallpox virus. In certain embodiments, the described DNAzymes are engineered to target expressed sequences of a virus, thus ameliorating viral activity and replication. In other embodiments, the DNAzymes are used in the treatment and/or diagnosis of viral infected tissue. In still other embodiments, the DNAzyme are used in treating virus-associated carcinoma, such as hepatocellular cancer.

In some embodiments, the RNA transcript is from a prokaryotic gene (e.g., a bacterial gene). In some embodiments, the RNA transcript is from a eukaryotic gene (e.g., a human gene). In some embodiments, the RNA transcript is a messenger RNA (mRNA) or a pre-messenger RNA (pre-mRNA) of a eukaryotic gene. In some embodiments, the RNA transcript is a P21 messenger RNA (mRNA). In other embodiments, the RNA transcript is a Ubiquitin-specific-processing protease 7 (USP7) messenger RNA (mRNA). In other embodiments, the RNA transcript is a Kirsten rat sarcoma virus (KRAS) messenger RNA (mRNA). In still other embodiments, the RNA transcript is a Baculoviral IAP Repeat Containing 5 (BIRC5) messenger RNA (mRNA).

In certain embodiments, the pharmaceutical compositions, guanine-rich DNAzymes, nucleic acids, or vectors described herein can be administered to a subject in need.

In certain embodiments, the pharmaceutical compositions, guanine-rich DNAzymes, nucleic acids, or vectors described herein can be administered in conjunction with any other conventional treatments. These treatments may be applied as necessary and/or as indicated and may occur before, concurrent with or after administration of the pharmaceutical compositions, DNAzymes, nucleic acids, vectors, dosage forms, or kits described herein.

In certain embodiments, the method comprises the administration of multiple doses of the guanine-rich DNAzyme, nucleic acid, or vector. Separate administrations can include any number of 2 or more administrations (e.g., doses), including 2, 3, 4, 5, 6, 7, 8, 9, 10, 11, 12, 13, 14, 15, 16, 20, 21, 22, 23, 24, or 25 administrations. In some embodiments, at least 8, 9, 10, 11, 12, 13, 14, or 15 administrations are included. One skilled in the art can readily determine the number of administrations to perform, or the desirability of performing one or more additional administrations, according to methods known in the art for monitoring therapeutic methods and other monitoring methods provided herein. Accordingly, the methods provided herein include methods of providing to the subject one or more administrations of a guanine-rich DNAzyme, a nucleic acid, a vector and/or a pharmaceutical composition described herein, where the number of administrations can be determined by monitoring the subject, and based on the results of the monitoring, determining whether or not to provide one or more additional administrations. Deciding on whether or not to provide one or more additional administrations can be based on a variety of monitoring results, including, but not limited to, indication of cell growth or inhibition of cell growth, cleavage of the target RNA transcripts, expression level (e.g., mRNA and/or protein level) of the target gene, the overall health of the subject and/or the weight of the subject.

The time period between administrations can be any of a variety of time periods. In some embodiments, the doses may be separated by at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days or 1, 2, 3, or 4 weeks. The time period between administrations can be a function of any of a variety of factors, including monitoring steps, as described in relation to the number of administrations, the time period for a subject to mount a response and/or the time period for a subject to clear the guanine-rich DNAzymes, nucleic acids, or vectors from normal tissue.

The administered dose of a guanine-rich DNAzyme, a nucleic acid, or a vector described herein is the amount of the guanine-rich DNAzyme, nucleic acid, or vector that is effective to achieve the desired therapeutic response for a particular patient, composition, and mode of administration, with the least toxicity to the patient or the maximal feasible dose. The effective dosage level can be identified using the methods described herein and depends upon a variety of pharmacokinetic factors including the activity of the particular compositions administered, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. In general, an effective dose of a therapy is the amount of the therapeutic agent which is the lowest dose effective to produce a therapeutic effect. Such an effective dose generally depends upon the factors described above.

Examples of routes of administration include oral administration, rectal administration, topical administration, inhalation (nasal) or injection. Administration by injection includes intravenous (IV), intraperitoneal, intranasal, intraarterial, intravesicle, intraocular, transdermal intralesional, intramuscular (IM), and subcutaneous (SC) administration. The compositions described herein can be administered in any form by any effective route, including but not limited to oral, parenteral, enteral, intravenous, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), implanted, intravesical, intrapulmonary, intraduodenal, intragastrical, and intrabronchial. In some embodiments, the guanine-rich DNAzymes, nucleic acids, vectors, or pharmaceutical compositions described herein are administered orally, rectally, topically, intravesically, by injection into or adjacent to a draining lymph node, intravenously, by inhalation or aerosol, or subcutaneously. In some embodiments, the administration is parenteral administration (e.g., subcutaneous administration).

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, general health and specific biomarkers, duration and route of administration, the kind and stage of the disease, and other compounds such as drugs being administered concurrently.

The dose of the pharmaceutical compositions described herein may be appropriately set or adjusted in accordance with the dosage form, the route of administration, the degree or stage of a target disease, and the like.

One skilled in the art will recognize that dosage will depend upon a variety of factors including the strength of the particular compound employed, as well as the age, species, condition, and body weight of the subject. The size of the dose will also be determined by the route, timing, and frequency of administration as well as the existence, nature, and extent of any adverse side-effects that might accompany the administration of a particular compound and the desired physiological effect.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. An effective dosage and treatment protocol can be determined by routine and conventional means, starting, e.g., with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including humans.

In accordance with the above, in therapeutic applications, the dosages of the guanine-rich DNAzymes or vectors provided herein may vary depending on the specific guanine-rich DNAzyme or vector, the age, weight, and clinical condition of the recipient patient, and the experience and judgment of the clinician or practitioner administering the therapy, among other factors affecting the selected dosage.

The articles “a” and “an” are used herein to refer to one or to more than one (e.g., to at least one) of the grammatical object of the article. By way of example, “an element” means one element or more than one element.

In one embodiment, the term “about”, refers to a deviance of between 0.0001-5% from the indicated number or range of numbers. In one embodiment, the term “about”, refers to a deviance of between 1-10% from the indicated number or range of numbers. In one embodiment, the term “about”, refers to a deviance of up to 25% from the indicated number or range of numbers.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

In some embodiments, “treatment” may encompass both therapeutic treatment and prophylactic or preventative measures, wherein the object is to prevent or lessen the targeted pathologic condition or disorder as described hereinabove. Thus, in one embodiment, treating may include directly affecting or curing, suppressing, inhibiting, preventing, reducing the severity of, delaying the onset of, reducing symptoms associated with the disease, disorder or condition, or a combination thereof, for example but not limited to treating cancer. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

Unless otherwise defined, all technical and/or scientific terms used herein have the same meaning as commonly understood by one of ordinary skill in the art. Although methods and materials similar or equivalent to those described herein can be used in the practice or testing of embodiments of the guanine-rich DNAzymes, non-limiting examples of methods and/or materials are described below. In case of conflict, the patent specification, including definitions, will control. In addition, the materials, methods, and examples are illustrative only and are not intended to be necessarily limiting.

### EXAMPLES

### Example 1: Functional DNAzyme Screen to Identify Efficient DNAzymes

Methods: The data gathered here was analyzed for features in the DNAzyme sequence that correlate with cell killing, as described below. A screen of DNAzymes targeted to the p21 transcript, a gene involved in regulation of cellular senescence, was analyzed. The dataset screened consisted of 558 DNAzymes, among them 222 DNAzymes of type 10-23.

The in vitro cleavage assay was performed by diluting RNA substrate with Molecular Biology Grade Water (Biological Industries). A 20 μl reaction system contained RNA substrate (final concentration 500 nM), 2 μl 10× reaction buffer (50 mM Tris-HCl pH 7.4, 100 mM NaCl, 10 mM MgCl₂) and 1 μl DNAzyme (10 μM). Reactions were incubated at 37° C. for 60 min. 2×RNA LD (Thermo Fisher) was added to each sample. Samples were heated at 70° C. for 30 sec, centrifuged and loaded onto 1.5% agarose TBE gels. Quantification was performed using Image Lab software (Bio Rad).

The in vivo screen comprised a cell death induction assay in 2D cultures of senescent cells transfected with the DNAzyme, wherein the cells are dependent on p21 expression for survival. The full group of DNAzymes recognizing the p21 sequence were screened. The DNAzymes screened comprised first and second substrate-binding regions having varied nucleotide sequences, both as to length of the region and percent (%) G.

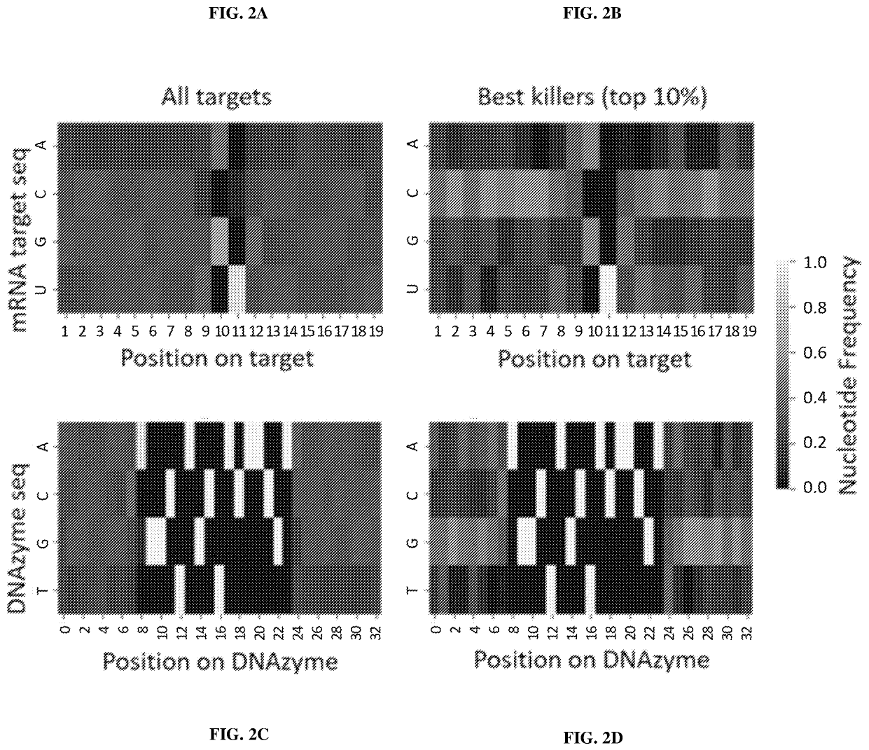

Results: The initial set of DNAzymes screened were not biased towards specific nucleotide sequence along the base-pairing arms. Focusing on the 10% most efficient 10-23 DNAzymes (Table 4), an enrichment of cytosine was found in the target mRNA sequence, hence guanine in the DNAzymes, uniformly along the arms (FIGS. 2A-2D). Continuing further with 10-23 DNAzymes, it was found that the G (guanine) content correlated with efficient induction of cell death, where the three other nucleotides showed mild-negative correlation (FIG. 3).

TABLE 4
Representative sequences of most efficient
10-23 DNAzymes.

| Sequence | SEQ ID NO. |
|---|---|
| AGGAGAACAGGCTAGCTACAACGAGGGATGAGG | 39 |
| TGCAATGAAGGCTAGCTACAACGATGGGGAGGG | 41 |
| TTCACAAGAGGCTAGCTACAACGAAGAGGGGGG | 42 |
| CCGTATACAGGCTAGCTACAACGATGCTGGGGA | 43 |
| GGGAAAGGAGGCTAGCTACAACGAAAGGGGGAG | 44 |
| AGGGGAGGAGGCTAGCTACAACGATTGACGAGT | 45 |
| AGGGGGGTAGGCTAGCTACAACGACAAGAGCCA | 46 |
| GGGGAGGGAGGCTAGCTACAACGAGGGGTGGAT | 47 |
| GAGGGGCCAGGCTAGCTACAACGAGAGGGCAGG | 48 |
| AAAGTGCAAGGCTAGCTACAACGAGAACTGGGG | 49 |

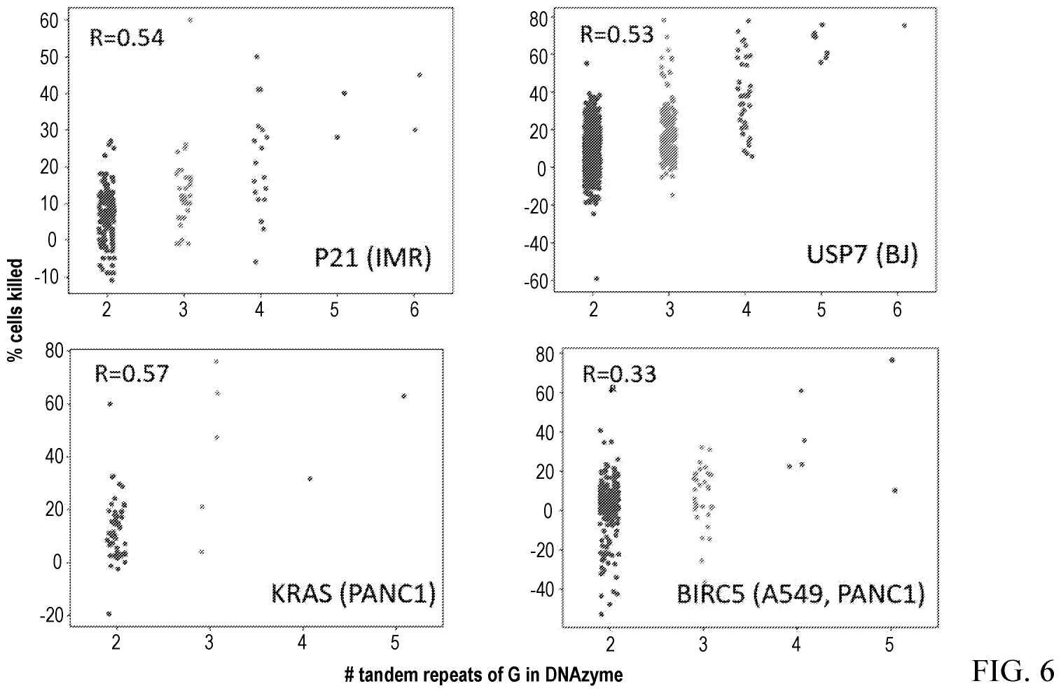

As G content, but not C content, correlated with DNAzyme killing efficiency, mild-positive or no correlation of killing efficiency with melting temperature (Tm) was expected, as the latter depends equally on C and G content. Indeed, these features showed a mild but positive correlation coefficient (FIG. 4). Furthermore, G-rich DNAzymes showed a tendency for higher cleavage efficiency in vitro (FIG. 5) when tested with the p21 transcript.

These results indicated that the guanine content of DNAzyme arms is correlated with DNAzyme killing efficiency, for example when targeted to a p21 transcript. Tandem repeats of guanine were associated with even better killing efficiency.

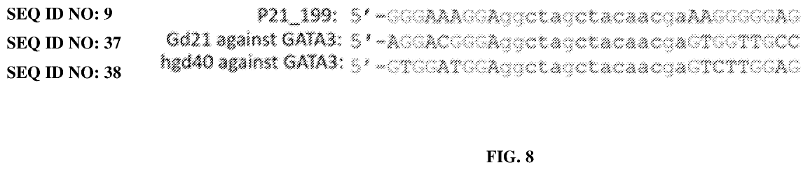

To confirm these findings, the maximal length of a tandem repeat stretch per DNAzyme was quantified for four (4) different targets: P21, USP7, KRAS, and BIRC5 (FIG. 6). Correlations were also seen between cell death vs. the number of tandem repeats in the 5′ and 3′ arms (FIG. 7; upper left and right panels); and between cell death vs. G content in the 5′ and 3′ arms (FIG. 7; lower left and right panels).

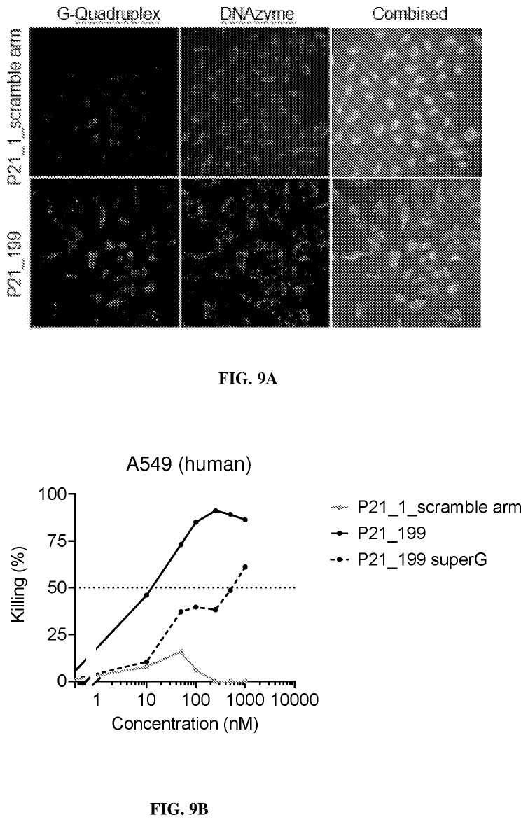

A strong positive correlation of G repeats and killing efficiency was found for all 4 gene targets (representative efficient DNAzyme sequences of for USP7, KRAS, and BIRC5 DNAzymes analyzed in FIG. 6 are shown in Table 5). This trend was consistent across all 4 tested targets and also in viability assays in multiple cell lines, including cancerous cell lines (A549, HepG2, and Panc-1). Furthermore, P21_199 (SEQ ID NO: 9), a highly efficient DNAzyme, was G-enriched. A retrospective analysis identified similar characteristics in clinically tested DNAzymes gd21 and hgd40, which target GATA3 (FIG. 8).

TABLE 5
Representative efficient DNAzyme sequences for other targets.

| Target | Sequence | SEQ ID NO. |
|---|---|---|
| USP7 | AGGGAAGGGGGCTAGCTACAACGACAGGTCCGC | 50 |
| USP7 | GAGGAAGGGGGCTAGCTACAACGACGACAATGC | 51 |
| USP7 | GCTGCAGGGGGCTAGCTACAACGATGGTGCACG | 52 |
| USP7 | TCACACGGGGGCTAGCTACAACGACACAAGGCA | 53 |
| USP7 | CCAGGAAGGGGCTAGCTACAACGAAGCCGAGCC | 54 |
| USP7 | AGGGCCAGGGGCTAGCTACAACGACCGCGGGGA | 55 |
| USP7 | CAGGGCTGGGGCTAGCTACAACGAGCACGGGAC | 56 |
| USP7 | CCCGAAGGGGGCTAGCTACAACGACTCGTGACA | 57 |
| KRAS | AGTCCGAAAGGCTAGCTACAACGAGGCGGGGGC | 58 |
| KRAS | GGACCGGGAGGCTAGCTACAACGATATGTCTCT | 59 |
| KRAS | TGTCGCTAAGGCTAGCTACAACGAGGATTGGGC | 60 |
| KRAS | TTAGCTGTAGGCTAGCTACAACGACGTCAAGGC | 61 |
| KRAS | GGGGAGAGAGGCTAGCTACAACGAGGGCCCTCA | 62 |
| KRAS | GGTCGTATAGGCTAGCTACAACGACAAAGGCCT | 63 |
| KRAS | TGTCGAGAAGGCTAGCTACAACGAATCCAAGAG | 64 |
| KRAS | GGTCCCTCAGGCTAGCTACAACGATGCACTGTA | 65 |
| BIRC5 | AGGGGGCAAGGCTAGCTACAACGAGTCGGGGCA | 66 |
| BIRC5 | AAGCGGGGAGGCTAGCTACAACGACTGGGCGGA | 67 |
| BIRC5 | GGGTCGTCAGGCTAGCTACAACGACTGGCTCCC | 68 |
| BIRC5 | ATGCATCTAGGCTAGCTACAACGACAAAAAAAA | 69 |
| BIRC5 | CCTCGGCCAGGCTAGCTACAACGACCGCTCCGG | 70 |
| BIRC5 | GCTGCTCGAGGCTAGCTACAACGAGGCACGGCG | 71 |
| BIRC5 | CAGGTGTGAGGCTAGCTACAACGAAGATAAGGA | 72 |
| BIRC5 | GTTCCTCTAGGCTAGCTACAACGAGGGGTCGTC | 73 |

G-quadruplexes (G-quads) are enriched in genomic regions such as telomeres and cis-regulatory elements, and are known to affect DNA stability and shape. Examining some of the DNAzyme sequences in a G-quad prediction tool (Kikin et al. (2006) Nucleic Acids Research 34:W676-W682) revealed a strong tendency of P21_199 (SEQ ID NO: 9 to fold in a G-quad (G-score=7), while for the scramble control (SEQ ID NO: 16), no G quads were found) (Table 6).

TABLE 6
QGRS output: Possibilities for G-quadruplex formation for p21_199
DNAzyme sequences. Nucleotides of the arms and the catalytic loop
are in CAPS and lower case letters, respectively. Underscored
nucleotides were identified by the program as able to form a G-quad.

| | | | G- |
|---|---|---|---|
| Length | QGRS | SEQ ID NO: | Score |
| 33 | GGGAAAGGAggctagctacaacgaAAGGGGGAG | 9 | 7 |
| 25 | GGAggctagctacaacgAAAGGGGG | 15 | 5 |
| 33 | ATCTTCCTTggctagctacaacgaCGCCTCTCC | 16 | 0 |
| 28 | GGGAAAGGAggctagctacaacgAAAGG | 17 | 7 |
| 29 | GGGAAAGGAggctagctacaacgAAAGGG | 18 | 6 |
| 30 | GGGAAAGGAggctagctacaacgAAAGGGG | 19 | 5 |
| 30 | GGGAAAGGAggctagctacaacgAAAGGGG | 20 | 3 |
| 30 | GGGAAAGGAggctagctacaacgAAAGGGG | 21 | 6 |
| 27 | GGAAAGGAggctagctacaacgAAAGG | 22 | 7 |
| 28 | GGAAAGGAggctagctacaacgAAAGGG | 23 | 6 |
| 29 | GGAAAGGAggctagctacaacgAAAGGGG | 24 | 5 |
| 29 | GGAAAGGAggctagctacaacgAAAGGGG | 25 | 3 |
| 29 | GGAAAGGAggctagctacaacgAAAGGGG | 26 | 6 |
| 30 | GGAAAGGAggctagctacaacgAAAGGGGG | 27 | 4 |
| 30 | GGAAAGGAggctagctacaacgAAAGGGGG | 28 | 4 |
| 30 | GGAAAGGAggctagctacaacgAAAGGGGG | 29 | 2 |
| 30 | GGAAAGGAggctagctacaacgAAAGGGGG | 30 | 7 |
| 30 | GGAAAGGAggctagctacaacgAAAGGGGG | 31 | 5 |
| 24 | GGAggctagctacaacgAAAGGGG | 32 | 6 |
| 25 | GGAggctagctacaacgAAAGGGGG | 33 | 7 |

Summary: The results presented here demonstrate that DNAzymes comprising targeting arms enriched for guanine nucleotides, and especially guanine tandem repeats, show an increase in cell death induction.

### Example 2: Evaluation of G-Quadruplex Contribution to Guanine-Rich DNAzymes Efficacy Killing

Objective: To evaluate the contribution of G-quadruplex formation in guanine-rich DNAzymes cytotoxic activity.

### Methods:

### Immunofluorescence Staining

Human IMR-90 cells were obtained from ATCC and maintained in DMEM 100 units per ml of penicillin, 100 mg/ml of streptomycin and 10% fetal bovine serum at 37° C., 5% CO₂ and 5% O₂. Cells were plated in 24-well plates at 100,000 cells per well, and transfected with 500 nM Cy5-labeled DNAzymes using cationic-lipid transfection reagent sold under the trademark Lipofectamine™ 2000 (11668-019, Invitrogen™) according to manufacturer instructions. 4 hours after transfection, cells were fixed in 4% paraformaldehyde/PBS and permeabilized with 0.1% Triton-X100/PBS. After blocking in 2% bovine serum albumin (BSA), immunofluorescence was performed using standard methods with BG4 antibody (Biffi et al. 2013; Nat Chem. 2013 March; 5(3):182-6. doi: 10.1038/nchem.1548), and Hoechst (H21486, Thermo) counterstaining. Cells were washed with PBS three times following each step. Images were acquired using Nikon Eclipse Ti2 microscope at ×10 magnification.

### Killing Assays

Human A549 lung cancer cell line and mouse Neuro-2a neuroblast cell line were obtained from ATCC. A549 were maintained in medium and Neuro-2a were maintained in medium (F-12K and EMEM, correspondingly) supplemented with 100 units per ml of penicillin, 100 mg/ml of streptomycin and 10% FBS at 37° C. and 5% CO₂. Cells were plated in 96-well plates at 15,000 cells per well and transfected with different concentrations of DNAzymes using Lipofectamine™ 2000, according to the manufacturer's instructions. Percentage of killing was determined based on quantification of remaining adherent cells using a resazurin-based solution sold under the trademark PrestoBlue™ (A13262, Life Technologies Ltd.) relative to control Lipofectamine™ alone treated cells, 3 days following transfection.

### P21_199 SuperG at Locations 2+11+13

The guanine-rich DNAzyme, P21_199 (SEQ ID NO: 9) was modified to incorporate a modified version of the base guanine (G) at positions 1, 11, and 13, counting from the 5′ end. A 8-aza-7-deazaguanosine replaced the guanine at these position. The modification is often termed a SuperG modification, wherein the modified base eliminates naturally occurring, non-Watson-and-Crick secondary structures associated with guanine-rich sequences. The resultant sequence of the P21_199 SuperG DNAzyme is set forth in SEQ ID NO: 34: G/iSuper-dG/GAAAGGAGGCTAGCTACAACGAAAG/iSuper-dG/G/iSuper-dG/GAG, wherein “iSuper-dG” represents an 8-aza-7-deazaguanosine.

### Results:

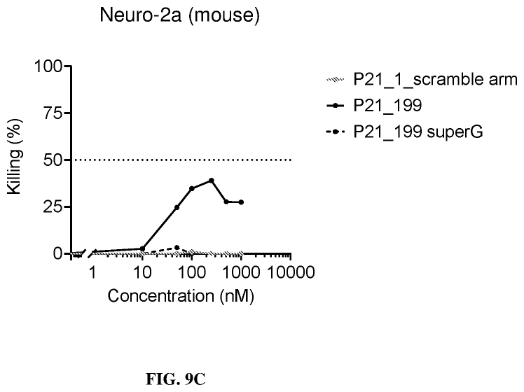

FIG. 9A demonstrates the colocalization of guanine-rich DNAzyme P21_199 and G-Quadruplex structure, wherein no G-Quadruplex structure is observed in the scrambled DNAzyme. FIGS. 9B and 9C demonstrate the increased efficacy of cell death induction by the guanine-rich DNAzyme comprising G-Quadruplex structure, compared with the SuperG DNAzyme in which the G-Quadruplex does not form.

Summary: The presence of G-Quadruplex structure within a DNAzyme increases the killing efficiency of the DNAzyme in vitro.

### Example 3: Enhancement of Cell Death Induction by DNAzymes with Guanine-Rich Overhangs

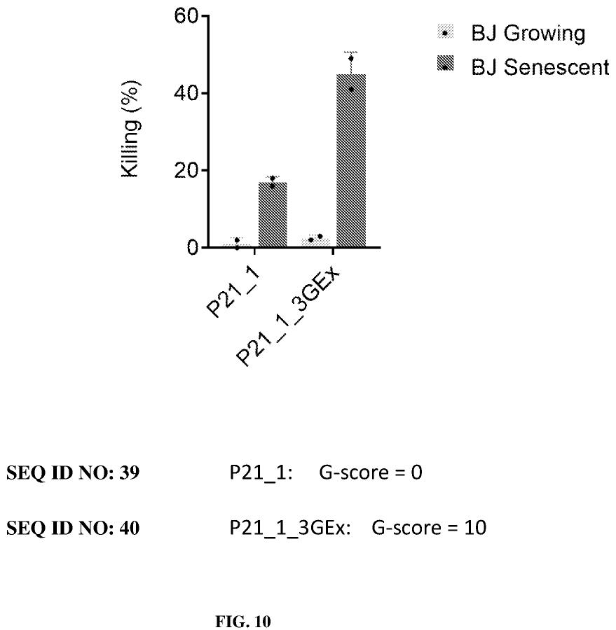

A cell death induction assay was performed in human fibroblast BJ cells, using a DNAzyme from Example 1 (SEQ ID NO. 39) vs. the same sequence, but adding a CGGG 3′ overhang (AGGAGAACAggctagctacaacgaGGGATGAGGCGGG; SEQ ID NO. 40). An increase in toxicity, specific for senescent cells, was observed (FIG. 10). Other DNAzymes also exhibited increased toxicity upon addition of G-rich overhangs.

While certain features of the guanine-rich DNAzymes have been illustrated and described herein, many modifications, substitutions, changes, and equivalents will now occur to those of ordinary skill in the art. It is, therefore, to be understood that the appended claims are intended to cover all such modifications and changes as fall within the true spirit of the guanine-rich DNAzymes disclosed herein.

What is claimed is:

1. A DNAzyme targeting a RNA transcript, the DNAzyme comprising, in 5′ to 3′ order:
(i) a first substrate-binding domain comprising a sequence that base pairs with a first region of the RNA transcript;
(ii) a DNAzyme catalytic core; and
(iii) a second substrate-binding domain comprising a sequence that base pairs with a second region of the RNA transcript positioned 5′ to the first region of the RNA transcript, said DNAzyme further comprising at least one of
(a) a 5′ overhang sequence having at least 50% G content, 5′ to said first substrate binding domain, or
(b) a 3′ overhang sequence having at least 50% G content, 3′ to said second substrate binding domain;
wherein upon binding of the DNAzyme to the RNA transcript, the DNAzyme catalytic core cleaves the RNA transcript at a position between the first and second region of the RNA transcript.

2. The DNAzyme of claim 1, wherein the DNAzyme catalytic core is a 10-23 catalytic core (SEQ ID NO: 1), a 8-17 catalytic core (SEQ ID NO: 2), a E1111 catalytic core (SEQ ID NO: 3), a E2112 catalytic core (SEQ ID NO: 4), a E5112 catalytic core (SEQ ID NO: 5), or a bipartite catalytic core (SEQ ID NO: 6).

3. The DNAzyme of claim 1, wherein the first substrate-binding domain or the second substrate-binding domain or both the first and the second substrate binding domains are 6-15 nucleotides in length.

4. The DNAzyme of claim 1, wherein: (a) the first substrate-binding domain is 100% complementary to the first region of the RNA transcript or partially complementary to the first region of the RNA transcript with no more than two mismatches: (b) the second substrate-binding domain is 100% complementary to the second region of the RNA transcript or partially complementary to the second region of the RNA transcript with no more than two mismatches; and (c) the first substrate-binding domain and the second substrate-binding domain together have no more than 3 mismatches to the first and second regions of the RNA transcript.

5. The DNAzyme of claim 1, wherein said RNA transcript is from a prokaryotic gene.

6. The DNAzyme of claim 1, wherein said RNA transcript is a messenger RNA (mRNA) or a pre-messenger RNA (pre-mRNA) of a eukaryotic gene.

7. The DNAzyme of claim 6, wherein said mRNA is selected from a p21 mRNA, an USP7 mRNA, a KRAS mRNA, and a BIRC5 mRNA.

8. The DNAzyme of claim 1, wherein said DNAzyme comprises both a 5′ overhang sequence having at least 50% G content and a 3′ overhang sequence having at least 50% G content.

9. The DNAzyme of claim 8, wherein said 5′ overhang sequence and said 3′ overhang sequence each have at least 1 tandem G repeat.

10. The DNAzyme of claim 1, wherein at least 30% of the nucleotides in the first substrate-binding domain and/or the second substrate-binding domain are guanine (G).

11. The DNAzyme of claim 1, wherein at least one of the 5′ overhang and the 3′ overhang is 1-10 nucleotides in length.

12. The DNAzyme of claim 1, wherein at least one of the first substrate-binding domain and the second substrate-binding domain comprises at least one tandem repeat of G of two or more consecutive Gs.

13. The DNAzyme of claim 1, wherein the 5′ extension and the 3′ extension form a structure within the extension itself, or form a structure with each other; wherein the structure is a structure formed between DNA strands based on Hoogsteen or wobble base pairing, or wherein the structure is a G-quadruplex, G-triplex, or H-DNA.

14. The DNAzyme of claim 1, wherein the DNAzyme comprises a chemical modification selected from a base modification, a sugar modification, and an internucleotide linkage modification.

15. The DNAzyme of claim 1, wherein the DNAzyme is chemically modified with poly-ethylene glycol (PEG) and or is modified with a cholesterol or a dialkyl lipid, wherein the cholesterol or diakyl lipid is linked to the 5′ end, the 3′ end, or both ends of the DNAzyme.

16. (canceled)

17. The DNAzyme of claim 1, wherein the DNAzyme is linked to a cell penetration-enhancing moiety.

18. A nucleic acid comprising one or more DNAzymes of claim 1 or the complementary sequences of one or more DNAzymes of claim 1.

19. A vector comprising the nucleic acid of claim 18.

20. A pharmaceutical composition, comprising the vector of claim 19 and a pharmaceutically acceptable carrier.

21. (canceled)

22. (canceled)

23. (canceled)

24. A method of cleaving a RNA transcript, comprising contacting the RNA transcript with a DNAzyme of claim 1.

25. A method of inhibiting expression of a gene, said method comprising contacting a RNA transcript of the gene with a DNAzyme of claim 1.

26. A method of treating cancer comprising administering a composition comprising a DNAzyme of claim 1.

27. (canceled)
